(19) United States
(12) Patent Application Publication
(10) Pub. No.: US 2011/0237609 A1
(43) Pub. Date: Sep. 29, 2011

(54) ANTIFOLATE COMPOSITIONS

(75) Inventor: Harish K. Pimplaskar, Charlotte, NC (US)

(73) Assignee: Chelsea Therapeutics, Inc.

(21) Appl. No.: 13/070,859

(22) Filed: Mar. 24, 2011

Related U.S. Application Data
(60) Provisional application No. 61/318,416, filed on Mar. 29, 2010.

(51) Int. Cl. A61K 31/517; A61P 35/00; A61P 29/00; A61P 11/06

(52) U.S. Cl. 514/266.4

(57) ABSTRACT

The present invention provides pharmaceutical compositions comprising an antifolate compound. The compositions can be prepared such that unit dosages of the composition exhibit excellent API content uniformity, such as by wet granulation or hot melt granulation. The pharmaceutical compositions are useful in the treatment of multiple conditions, including abnormal cell proliferation, inflammatory diseases, asthma, and arthritis.

### CROSS-REFERENCE TO RELATED APPLICATIONS

The present application claims priority to U.S. Provisional Patent Application No. 61/318,416, filed Mar. 29, 2010, the complete disclosure of which is incorporated herein by reference in its entirety.

### FIELD OF THE INVENTION

The present application is directed to pharmaceutical compositions comprising active compounds and methods of preparing such compositions. More specifically, the pharmaceutical compositions comprise antifolate compounds.

### BACKGROUND

Folic acid is a water-soluble B vitamin known by the systematic name N-[4(2-amino-4-hydroxy-pteridin-6-ylmethylamino)-benzoyl]-L(+)-glutamic acid and having the structure provided below in Formula (1).

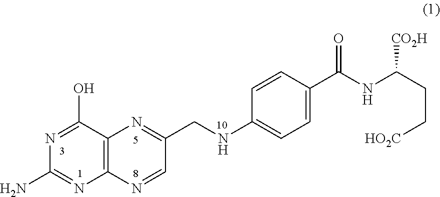

As seen in Formula (1), the folic acid structure can generally be described as being formed of a pteridine ring, a para-aminobenzoic acid moiety, and a glutamate moiety. Folic acid and its derivatives are necessary for metabolism and growth, particularly participating in the body's synthesis of thymidylate, amino acids, and purines. Derivatives of folic acid, such as naturally occurring folates, are known to have biochemical effects comparable to folic acid. Folic acid is known to be derivatized via hydrogenation, such as at the 1,4-diazine ring, or being methylated, formaldehydylated, or bridged, wherein substitution is generally at the N⁵ or N¹⁰ positions. Folates have been studied for efficacy in various uses including reduction in severity or incidence of birth defects, heart disease, stroke, memory loss, and age-related dementia.

Antifolate compounds, like folates, are structurally similar to folic acid; however, antifolate compounds function to disrupt folic acid metabolism. A review of antifolates is provided by Takamoto (1996) The Oncologist, 1:68-81, which is incorporated herein by reference. One specific group of antifolates, the so-called “classical antifolates,” is characterized by the presence of a folic acid p-aminobenzoylglutamic acid side chain, or a derivative of that side chain. Another group of antifolates, the so-called “nonclassical antifolates,” are characterized by the specific absence of the p-aminobenzoylglutamic group. Because antifolates have a physiological effect that is opposite the effect of folic acid, antifolates have been shown to exhibit useful physiological functions, such as the ability to destroy cancer cells by causing apoptosis.

Folate monoglutamylates and antifolate monoglutamylates are transported through cell membranes either in reduced form or unreduced form by carriers specific to those respective forms. Expression of these transport systems varies with cell type and cell growth conditions. After entering cells most folates, and many antifolates, are modified by polyglutamylation, wherein one glutamate residue is linked to a second glutamate residue at the a carboxy group via a peptide bond. This leads to formation of poly-L-γ-glutamylates, usually by addition of three to six glutamate residues. Enzymes that act on folates have a higher affinity for the polyglutamylated forms. Therefore, polyglutamylated folates generally exhibit a longer retention time within the cell.

An intact folate enzyme pathway is important to maintain de novo synthesis of the building blocks of DNA, as well as many important amino acids. Antifolate targets include the various enzymes involved in folate metabolism, including (i) dihydrofolate reductase (DHFR); (ii) thymidylate synthase (TS); (iii) folylpolyglutamyl synthase; and (iv) glycinamide ribonucleotide transformylase (GARFT) and aminoimidazole carboxamide ribonucleotide transformylase (AICART).

The reduced folate carrier (RFC), which is a transmembrane glycoprotein, plays an active role in the folate pathway transporting reduced folate into mammalian cells via the carrier mediated mechanism (as opposed to the receptor mediated mechanism). The RFC also transports antifolates, such as methotrexate. Thus, mediating the ability of RFC to function can affect the ability of cells to uptake reduced folates.

Polyglutamylated folates can function as enzyme cofactors, whereas polyglutamylated antifolates generally function as enzyme inhibitors. Moreover, interference with folate metabolism prevents de novo synthesis of DNA and some amino acids, thereby enabling antifolate selective cytotoxicity. Methotrexate, the structure of which is provided in Formula (2), is one antifolate that has shown use in cancer treatment, particularly treatment of acute leukemia, non-Hodgkin's lymphoma, breast cancer, head and neck cancer, choriocarcinoma, osteogenic sarcoma, and bladder cancer.

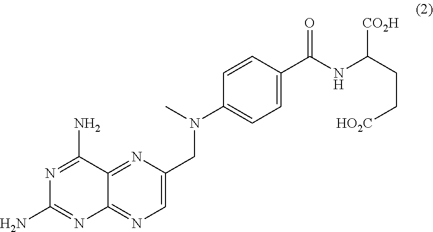

Nair et al. (J. Med. Chem. (1991) 34:222-227), incorporated herein by reference, demonstrated that polyglutamylation of classical antifolates was not essential for anti-tumor activity and may even be undesirable in that polyglutamylation can lead to a loss of drug pharmacological activity and target specificity. This was followed by the discovery of numerous nonpolyglutamylatable classical antifolates. See Nair et al. (1998) Proc. Amer. Assoc. Cancer Research 39:431, which is incorporated herein by reference. One particular group of nonpolyglutamylatable antifolates are characterized by a methylidene group (i.e., a ═CH₂ substituent) at the 4-position of the glutamate moiety. The presence of this chemical group has been shown to affect biological activity of the antifolate compound. See Nair et al. (1996) Cellular Pharmacology 3:29, which is incorporated herein by reference.

Further folic acid derivatives have also been studied in the search for antifolates with increased metabolic stability allowing for smaller doses and less frequent patient administration. For example, a dideaza (i.e., quinazoline-based) analog has been shown to avoid physiological hydroxylation on the pteridine ring system. Furthermore, replacement of the secondary amine nitrogen atom with an optionally substituted carbon atom has been shown to protect neighboring bonds from physiological cleavage.

One example of an antifolate having carbon replacement of the secondary amine nitrogen is 4-amino-4-deoxy-10-deazapteroyl-γ-methyleneglutamic acid—more commonly referred to as MDAM—the structure of which is provided in Formula (3).

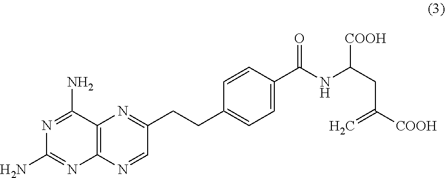

The L-enantiomer of MDAM has been shown to exhibit increased physiological activity. See U.S. Pat. No. 5,550,128, which is incorporated herein by reference. Another example of a classical antifolate designed for metabolic stability is ZD1694, which is shown in Formula (4).

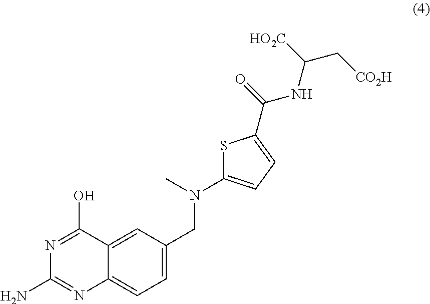

A group of antifolate compounds according to the structure shown in Formula (5) combines several of the molecular features described above, and this group of compounds is known by the names MobileTrexate, Mobile Trex, Mobiltrex, or M-Trex.

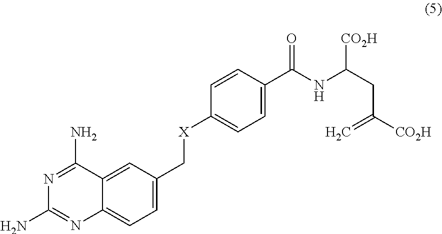

As shown in Formula (5), this group of compounds encompasses M-Trex, wherein X can be CH₂, CHCH₃, CH(CH₂CH₃), NH, or NCH₃.

The effectiveness of antifolates as pharmaceutical compounds arises from other factors in addition to metabolic inertness, as described above. The multiple enzymes involved in folic acid metabolism within the body present a choice of inhibition targets for antifolates. In other words, it is possible for antifolates to vary as to which enzyme(s) they inhibit. For example, some antifolates inhibit primarily dihydrofolate reductase (DHFR), while other antifolates inhibit primarily thymidylate synthase (TS), glycinamide ribonucleotide formyltransferase (GARFT), or aminoimidazole carboxamide ribonucleotide transformylase, while still other antifolates inhibit combinations of these enzymes.

In light of the usefulness of antifolates in treating a variety of conditions, there remains a need in the art for pharmaceutical compositions that can safely and effectively deliver the antifolates to a patient in need of treatment.

### SUMMARY OF THE INVENTION

The present invention provides pharmaceutical compositions comprising antifolate compounds. The pharmaceutical compositions provide the antifolate compounds in a form exhibiting excellent bioavailability. In specific embodiments, the antifolate compounds used in the compositions are in the form of salts. Such salts provide for improved solubility, particularly in lower pH ranges. The salt forms of the antifolate compounds are also beneficial for increasing the amount of the active compounds that is made available for biological activity when administered orally, even when the compositions comprise a reduced amount of the active antifolate compound. The compositions may be prepared by specific methods, such as wet granulation or hot melt granulation. Such specific methods are beneficial because the antifolate compound in particulate form may have varying size ranges that can adversely affect the per dosage content uniformity. The present invention has established compositions and methods of preparation thereof that provide not only the benefits described above but also excellent per dosage content uniformity. The pharmaceutical compositions of the invention are useful in the treatment of a variety of conditions including, but not limited to, abnormal cellular proliferation, asthma and other inflammatory diseases, and rheumatoid arthritis and other autoimmune diseases.

In one aspect, the present invention provides specific pharmaceutical compositions. Such compositions comprise at least one active pharmaceutical ingredient (API), preferably at least one antifolate compound. The compositions also may comprise additional ingredients that are useful to provide the compositions in forms that exhibit good bioavailability and good per dosage content uniformity of the API.

In one embodiment, the present invention provides a pharmaceutical composition comprising an antifolate compound according to Formula (6):

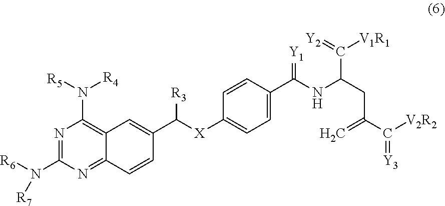

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkenyl; or a pharmaceutically acceptable ester, amide, salt, solvate, enantiomer, or prodrug thereof.

In specific embodiments, the pharmaceutical composition may comprise about 0.01% to about 20% by weight of the antifolate compound. Further, the antifolate compound can be in stable, particulate form having a defined range of particle sizes. In some embodiments, the antifolate particles may have a size range of about 10 μm to about 250 μm. Such size range has been found to be detrimental to the ability to successfully form compositions using the antifolate compounds. Without wishing to be bound by theory, it is believed that since the antifolate compounds in the present invention can be provided in particularly low, effective dosages, the relatively large particle sizes and the variability in particle sizes makes it difficult to maintain uniformity in API content between individual dosages. The present invention overcomes this problem, preferably without the need for size processing of the API, which can be costly and can be detrimental to the API itself.

In specific embodiments, the pharmaceutical compositions can comprise one or more pharmaceutically acceptable excipients. For example, the composition may comprise about 25% to about 95% by weight of at least one filler. In further embodiments, the composition may comprise about 1% to about 10% by weight of at lease one disintegrant. Still further, the composition may comprise about 0.1% to about 5% by weight of at least one lubricant. Of course, other excipients, such as described herein, also may be used in the inventive compositions. In certain embodiments, a pharmaceutical composition according to the invention may comprise microcrystalline cellulose as a filler and, preferably, may comprise at least two different types of microcrystalline cellulose. A separate or additional type of filler can be one that includes starch materials. Examples of disintegrants useful in the inventive pharmaceutical compositions include carboxymethyl cellulose and croscarmellose sodium, particularly combinations of both compounds. Examples of lubricants useful according to the invention include fatty acids or salts thereof. In one particular embodiment, a composition according to the invention may include microcrystalline cellulose, starch, croscarmellose sodium, and magnesium stearate as useful excipients.

Further to the above, the beneficial API content uniformity of the inventive compositions can be described in terms of the percent relative standard deviation (% RSD) of the API content between individual dosages (e.g., individual capsules or individual tablets). Thus, the composition of the invention may be described in terms of being provided as individual unit dosages. Preferably, a group of individual unit dosages exhibits a desirably low % RSD, such as further described herein. In other words, the invention makes it possible to provide a plurality of dosages that are substantially similar in API content such that the % RSD for the plurality of dosages is within a defined range or below a defined threshold. Such plurality of dosages may be an entire batch of dosages.

The present compositions and methods of preparation thereof are particularly beneficial in that any antifolate compounds encompassed by Formula (6) above may be used therein. In certain embodiments, however, specific antifolate compounds encompassed by Formula (6) may particularly be beneficial. For example, in some embodiments, the antifolate compound may comprise a compound according to Formula (7):

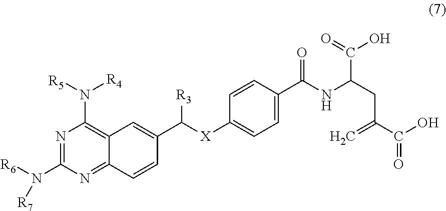

wherein:

X is CHR₈ or NR₈;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof.

In other embodiments, the antifolate compound used according to the invention may comprise a compound according to Formula (9):

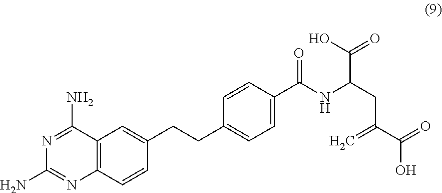

or a pharmaceutically acceptable ester, amide, salt, solvate, enantiomer, or prodrug thereof.

In still further embodiments, the antifolate compound used according to the invention may comprise a compound according to Formula (11):

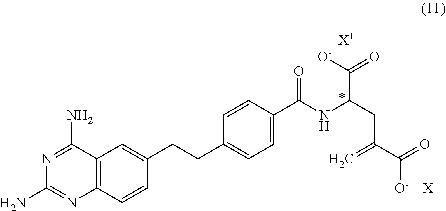

or an enantiomer thereof, wherein each X⁺ independently is a salt-forming counterion. In specific embodiments, X⁺ may be an alkali metal cation, such as sodium or potassium. Moreover, the antifolate compound particularly may be a crystalline salt. Further, the antifolate compounds of the invention may be in racemic form or may in a substantially enantiomerically purified form.

In other embodiments, the antifolate compound used according to the invention may comprise a compound according to Formula (12):

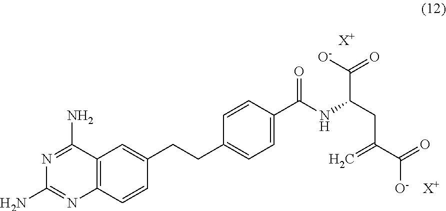

wherein each X⁺ independently is a salt-forming counterion, and wherein the antifolate compound is in the (S) enantiomeric form. Such compounds may exhibit specific levels of purity for the enantiomeric form, such as having an enantiomeric purity for the (S) enantiomer of at least about 90%.

In one specific embodiment, a pharmaceutical composition according to the invention can include an antifolate compound according to Formula (12) that is a crystalline, disodium salt in the (S) enantiomeric form exhibiting an enantiomeric purity for the (S) enantiomer of at least about 99%. In another specific embodiment, a pharmaceutical composition according to the invention can include an antifolate compound comprises a compound according to Formula (12) that is a crystalline, dipotassium salt in the (S) enantiomeric form exhibiting an enantiomeric purity for the (5) enantiomer of at least about 99%.

In another embodiment, a pharmaceutical composition according to the invention can comprise an alkali metal salt of (S)-2-{4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%. In specific embodiments, the composition may comprise about 0.01% to about 20% by weight of the compound. Further, the composition may comprise about 25% to about 95% by weight of at least one filler, about 1% to about 10% by weight of at least one disintegrant; and about 0.1% to about 5% by weight of at least one lubricant.

The invention also provides pharmaceutical compositions comprising further active agents. In particularly, the pharmaceutical composition can comprise one or more antifolate compounds as described herein in combination with one or more further active ingredients, such as methotrexate.

As noted above, the pharmaceutical compositions according to the invention may be prepared according to specific methods of preparation. In one embodiment, the composition may be prepared via wet granulation. In such embodiments, the wet granulation may comprise forming an intra-granular portion and blending the intra-granular portion with one or more extra-granular components. The antifolate compound particularly may be included in the intra-granular portion.

In another embodiment, pharmaceutical compositions according to the invention may be prepared via hot melt granulation. In such embodiments, the hot melt granulation may comprise forming a melt comprising the antifolate compound and one or more waxy excipients, such as polyglycolized glycerides.

Thus, in another aspect, the present invention provides methods of preparing the pharmaceutical compositions described herein. As noted herein, because of the physical nature of the compositions, particularly the particulate antifolate compounds used therein, it was found that composition preparation was particularly challenging, especially in relation to achieving an acceptable API content uniformity between individual dosage forms, as well as ensuring individual batches of the compositions achieved an acceptable mean API content. The present invention, however, identified useful methods for overcoming these difficulties.

In one embodiment, the invention thus provides a method of making a pharmaceutical composition comprising an antifolate compound according to Formula (6) comprising wet granulating the antifolate compound with one or more acceptable excipients. Any antifolate compound described herein may be used in such methods of preparation, particularly including any antifolate compound described and encompassed by any of the specific formulas provided herein. In specific embodiments, the method can comprise wet granulating the antifolate compound with at least one filler, at least one disintegrant, and at least one lubricant.

In a particular embodiment, a method according to the invention can comprise the following steps: a) forming a granule comprising the antifolate compound, at least one filler, a portion of a disintegrant, and a solvent; b) at least partially drying the granule; c) passing the granule through a granulator to form sized particles; and d) adding the remaining portion of the disintegrant and a lubricant with blending. In another embodiment, a method according to the invention can comprise the following steps: a) combining at least one filler and a portion of a disintegrant in a granulator; b) adding the antifolate compound dissolved in a suitable solvent; c) mixing to form a granule, optionally adding a further amount of solvent; d) at least partially drying the granule; e) passing the dried granule through a granulator fitted with a screen of desired size to form sized particles; and f) adding the remaining portion of the disintegrant and a lubricant with blending.

In another embodiment, the invention provides a method of making a pharmaceutical composition comprising an antifolate compound according to Formula (6) comprising hot melt granulating the antifolate compound with one or more acceptable excipients. Again, any antifolate compound described herein may be used in such methods of preparation. In some embodiments, the excipient can comprise a material that increases one or both of solubility and bioavailability of the antifolate compound. In particular, the excipient can comprise fatty acid esters of glycerol and polyethylene glycol esters and/or cyclodextrins. In certain embodiments, the excipient comprises GELUCIRE®, and particularly GELUCIRE® 44/14. In specific embodiments, the method may comprise hot melt granulating the antifolate compound with at least one filler, a polyglycolized glyceride, and a lubricant.

In some embodiments, the invention can provide methods of making a pharmaceutical composition comprising an antifolate compound according to Formula (6):

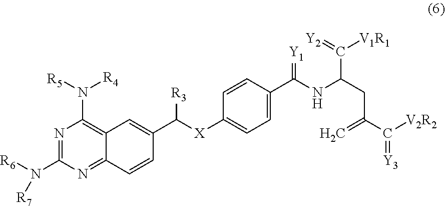

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;

the method comprising hot melt granulating about 0.01% to about 20% by weight of the antifolate compound with about 25% to about 95% by weight of at least one filler, about 5% to about 50% by weight of a polyglycolized glyceride; and about 0.1% to about 5% by weight of a lubricant.

More specifically, such hot melt granulation method may comprise the following steps: combining the antifolate compound with a filler; adding the polyglycolized glyceride under temperature conditions sufficient to fully liquefy the polyglycolized glyceride; mixing to form a granule; cooling the formed granule; and screening the granule and a lubricant into a container and blending the materials to form the composition.

In other embodiments, the materials used may exhibit specific characteristics and/or may be provided in defined ratios. For example, the polyglycolized glyceride may be characterized by one or more of the following: have a melting point that is less than about 50° C.; have an HLB value that is greater than about 8; comprise a C₁₄-C₂₀ fatty acid ester; and comprise a polyethylene glycol having a number average MW of about 1,200 to about 2,500 Da. Further, the polyglycolized glyceride and the antifolate compound may be present in a weight-to-weight ratio of about 1:1 to about 50:1. Likewise, the filler and the polyglycolized glyceride may be present in a weight-to-weight ratio of about 90:10 to about 50:50. Of course, such characteristics are only exemplary, and further characteristics and ratios as described herein also would be encompassed by the invention.

In a specific embodiment, a hot melt granulation method according to the invention may comprise the following steps: blending the antifolate compound with a first amount of the filler to form a pre-blend; combining the pre-blend with a second amount of the same or a different filler; adding the polyglycolized glyceride to the combined materials under temperature conditions sufficient to fully liquefy the polyglycolized glyceride; mixing the combination to form a granule; cooling the formed granule; screening the granule and a lubricant into a container; and blending the screened materials.

In further embodiments, the invention also can provide pharmaceutical compositions comprising about 0.01% to about 20% by weight of an antifolate compound according to Formula (6):

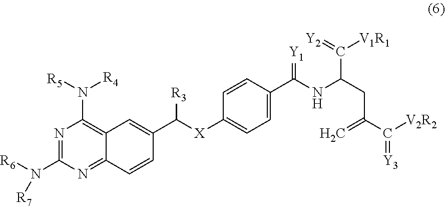

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkenyl; or a pharmaceutically acceptable ester, amide, salt, solvate, enantiomer, or prodrug thereof;

the antifolate compound being in particulate form having a range of particle sizes of about 10 μm to about 250 μm;

about 25% to about 95% by weight of at least one filler;

about 5% to about 50% by weight of a polyglycolized glyceride; and

about 0.1% to about 5% by weight of a lubricant.

The components of the composition in such embodiments may exhibit similar characteristics to those discussed herein in relation to the hot melt granulation method. For example, the polyglycolized glyceride can exhibit similar characteristics, and the materials may be combined in similar ratios.

In another aspect, the present invention also provides methods of treating various conditions. For example, in certain embodiments, the invention provides a method for treating a condition selected from the group consisting of abnormal cell proliferation, inflammation, asthma, and arthritis. Preferably, method comprising administering to a subject in need of treatment a pharmaceutical composition, such as described herein.

### DETAILED DESCRIPTION

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The invention provides pharmaceutical compositions comprising antifolate compounds. These compounds can be used in the pharmaceutical composition either directly or in the form of their pharmaceutically active esters, amides, salts, solvates, or prodrugs. In preferred embodiments, the antifolate compounds are in the form of salts, particularly alkali metal salts. The pharmaceutical compositions provide increased activity and bioavailability, even at reduced dosing of the active antifolate compounds, and the pharmaceutical compositions are useful in the treatment of a number of conditions and diseases, particularly for the treatment of abnormal cell proliferation, inflammation, arthritis, or asthma.

### I. DEFINITIONS

The term “active content uniformity” as used herein relates to the consistency of the content of an active pharmaceutical ingredient in a pharmaceutical composition. This term can be used in relation to individual dosage forms prepared from a given batch of a pharmaceutical composition comprising the API. “High active content uniformity” as used herein means the difference in API content between individual dosage forms is low. Dose active content uniformity can be evaluated in terms of the relative standard deviation of the content of the API in individual dosage forms prepared from a given batch of the pharmaceutical composition. Active content uniformity also may be used in relation to the ability to prepare different batches of the pharmaceutical composition that have a consistent API mean content (i.e., the average of the API content in the individual dosages prepared from the batch). Batch content uniformity can be evaluated as a comparison of the actual API mean content of a given batch in relation to the label strength, or the intended API mean content.

The term “metabolically inert antifolate” as used herein means compounds that are (i) folic acid analogs capable of disrupting folate metabolism and (ii) non-polyglutamylatable. In certain embodiments, the term can mean compounds that are also (iii) non-hydroxylatable.

The term “alkali metal” as used herein means Group IA elements and particularly includes sodium, lithium, and potassium; the term “alkali metal salt” as used herein means an ionic compound wherein the cation moiety of the compound comprises an alkali metal, particularly sodium, lithium, or potassium.

The term “alkyl” as used herein means saturated straight, branched, or cyclic hydrocarbon groups. In particular embodiments, alkyl refers to groups comprising 1 to 10 carbon atoms (“C₁₋₁₀ alkyl”). In further embodiments, alkyl refers to groups comprising 1 to 8 carbon atoms (“C₁₋₈ alkyl”), 1 to 6 carbon atoms (“C₁₋₆ alkyl”), or 1 to 4 carbon atoms (“C₁₋₄ alkyl”). In specific embodiments, alkyl refers to methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. Substituted alkyl refers to alkyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkenyl” as used herein means alkyl moieties wherein at least one saturated C—C bond is replaced by a double bond. In particular embodiments, alkenyl refers to groups comprising 1 to 10 carbon atoms (“C₁₋₁₀ alkenyl”). In further embodiments, alkenyl refers to groups comprising 1 to 8 carbon atoms (“C₁₋₈ alkenyl”), 1 to 6 carbon atoms (“C₁₋₆ alkenyl”), or 1 to 4 carbon atoms (“C₁₋₄ alkenyl”). In specific embodiments, alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Substituted alkenyl refers to alkenyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkynyl” as used herein means alkynyl moieties wherein at least one saturated C—C bond is replaced by a triple bond. In particular embodiments, alkynyl refers to groups comprising 1 to 10 carbon atoms (“C₁₋₁₀ alkynyl”). In further embodiments, alkynyl refers to groups comprising 1 to 8 carbon atoms (“C₁₋₈ alkynyl”), 1 to 6 carbon atoms (“C₁₋₆ alkynyl”), or 1 to 4 carbon atoms (“C₁₋₄ alkynyl”). In specific embodiments, alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl. Substituted alkynyl refers to alkynyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkoxy” as used herein means straight or branched chain alkyl groups linked by an oxygen atom (i.e., —O-alkyl), wherein alkyl is as described above. In particular embodiments, alkoxy refers to oxygen-linked groups comprising 1 to 10 carbon atoms (“C₁₋₁₀ alkoxy”). In further embodiments, alkoxy refers to oxygen-linked groups comprising 1 to 8 carbon atoms (“C₁₋₈ alkoxy”), 1 to 6 carbon atoms (“C₁₋₆ alkoxy”), or 1 to 4 carbon atoms (“C₁₋₄ alkoxy”). Substituted alkoxy refers to alkoxy substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “halo” or “halogen” as used herein means fluorine, chlorine, bromine, or iodine.

The term “aryl” as used herein means a stable monocyclic, bicyclic, or tricyclic carbon ring of up to 8 members in each ring, wherein at least one ring is aromatic as defined by the Hückel 4n+2 rule. Exemplary aryl groups according to the invention include phenyl, naphthyl, tetrahydronaphthyl, and biphenyl. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate.

The terms “aralkyl” and “arylalkyl” as used herein mean an aryl group as defined above linked to the molecule through an alkyl group as defined above.

The terms “alkaryl” and “alkylaryl” as used herein means an alkyl group as defined above linked to the molecule through an aryl group as defined above.

The term “acyl” as used herein means a carboxylic acid ester in which the non-carbonyl moiety of the ester group is selected from straight, branched, or cyclic alkyl or lower alkyl; alkoxyalkyl including methoxymethyl; aralkyl including benzyl; aryloxyalkyl such as phenoxymethyl; aryl including phenyl optionally substituted with halogen, C₁-C₆ alkyl or C₁-C₆ alkoxy; sulfonate esters such as alkyl or aralkyl sulphonyl including methanesulfonyl; mono-, di-, or triphosphate ester; trityl or monomethoxytrityl; substituted benzyl; trialkylsilyl such as dimethyl-t-butylsilyl or diphenylmethylsilyl. Aryl groups in the esters optimally comprise a phenyl group.

The term “amino” as used herein means a moiety represented by the structure NR₂, and includes primary amines, and secondary and tertiary amines substituted by alkyl (i.e., alkylamino). Thus, R₂ may represent two hydrogen atoms, two alkyl moieties, or one hydrogen atom and one alkyl moiety.

The terms “alkylamino” and “arylamino” as used herein mean an amino group that has one or two alkyl or aryl substituents, respectively.

The term “analogue” as used herein means a compound in which one or more individual atoms or functional groups have been replaced, either with a different atom or a different functional, generally giving rise to a compound with similar properties.

The term “derivative” as used herein means a compound that is formed from a similar, beginning compound by attaching another molecule or atom to the beginning compound. Further, derivatives, according to the invention, encompass one or more compounds formed from a precursor compound through addition of one or more atoms or molecules or through combining two or more precursor compounds.

The term “prodrug” as used herein means any compound which, when administered to a mammal, is converted in whole or in part to a compound of the invention.

The term “active metabolite” as used herein means a physiologically active compound which results from the metabolism of a compound of the invention, or a prodrug thereof, when such compound or prodrug is administered to a mammal.

The terms “therapeutically effective amount” or “therapeutically effective dose” as used herein are interchangeable and mean a concentration of a compound according to the invention, or a biologically active variant thereof, sufficient to elicit the desired therapeutic effect according to the methods of treatment described herein.

The term “pharmaceutically acceptable excipient” as used herein means an excipient that is conventionally used in the art to facilitate the storage, administration, and/or the healing effect of a biologically active agent.

The term “intermittent administration” as used herein means administration of a therapeutically effective dose of a composition according to the invention, followed by a time period of discontinuance, which is then followed by another administration of a therapeutically effective dose, and so forth.

The term “antiproliferative agent” as used herein means a compound that decreases the hyperproliferation of cells.

The term “abnormal cell proliferation” as used herein means a disease or condition characterized by the inappropriate growth or multiplication of one or more cell types relative to the growth of that cell type or types in an individual not suffering from that disease or condition.

The term “cancer” as used herein means a disease or condition characterized by uncontrolled, abnormal growth of cells, which can spread locally or through the bloodstream and lymphatic system to other parts of the body. The term includes tumor-forming or non-tumor forming cancers, and includes various types of cancers, such as primary tumors and tumor metastasis.

The term “tumor” as used herein means an abnormal mass of cells within a multicellular organism that results from excessive cell division that is uncontrolled and progressive, also called a neoplasm. A tumor may either be benign or malignant.

The term “fibrotic disorders” as used herein means fibrosis and other medical complications of fibrosis which result in whole or in part from the proliferation of fibroblasts.

The term “arthritis” as used herein means an inflammatory disorder affecting joints that can be infective, autoimmune, or traumatic in origin.

Chemical nomenclature using the symbols “D” and “L” or “R” and “S” are understood to relate the absolute configuration, or three-dimensional arrangement, of atoms or groups around a chiral element, which may be a center, usually an atom, an axis, or a plane. As used herein, the “D/L” system and the “R/S” systems are meant to be used interchangeably such that “D” in the former system corresponds to “R” in the later system and “L” in the former system corresponds to “S” in the later system.

### II. COMPOUNDS

The pharmaceutical compositions of the invention comprise one or more antifolate compounds. In specific embodiments, the antifolate compounds are metabolically inert antifolates. As recognized in the art, antifolates are compounds that interfere with various stages of folate metabolism. Thus, the compounds of the invention can particularly be used in pharmaceutical compositions useful for the treatment of diseases and conditions related to or capable of being treated by disruption of folate metabolism, or other biological mechanisms related to folate metabolism.

In one embodiment, the pharmaceutical compositions of the present invention comprise antifolate compounds having the structure provided in Formula (6),

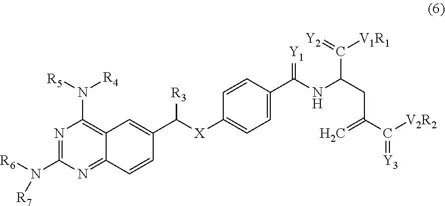

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; as well as pharmaceutically acceptable esters, amides, salts, solvates, enantiomers, and prodrugs thereof.

In another embodiment, the pharmaceutical compositions of the present invention comprise compounds having the structure provided in Formula (7)

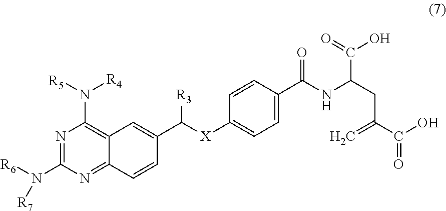

wherein:

X is CHR₈ or NR₈;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; as well as pharmaceutically acceptable esters, amides, salts, solvates, enantiomers, and prodrugs thereof.

In yet another embodiment, the pharmaceutical compositions of the present invention comprise antifolate compounds having the structure provided in Formula (8)

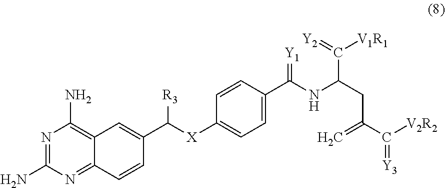

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₈ is H, optionally substituted alkyl, optionally substituted alkenyl, optionally substituted alkynyl, acyl, —C(O)-alkyl, —C(O)-alkenyl, or —C(O)-alkynyl as well as pharmaceutically acceptable esters, amides, salts, solvates, enantiomers, and prodrugs thereof.

In one particular embodiment, the present invention provides pharmaceutical compositions comprising an antifolate compound having the structure provided in Formula (9).

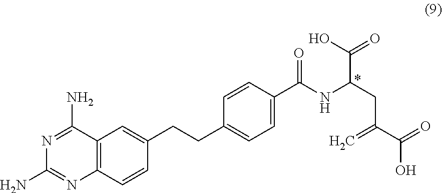

The compound of Formula (9) has been shown to have activity for the treatment of abnormal cellular proliferation, inflammation disorders, and autoimmune diseases. This compound may particularly be known by the name 2-{-4-[2-(2,4-diamino-quinazolin-6-yl)-ethyl]-benzoylamino}-4-methylene-pentanedioic acid. The compound may also be known as gamma methylene glutamate 5,8,10-trideaza aminopterin or 5,8-dideaza MDAM. The antifolate compound of Formula (9) is non-polyglutamylatable, non-hydroxylatable, and capable of disrupting folate metabolism. The compound has also shown effectiveness in killing large numbers of human leukemia cells and human solid tumor cells in culture at therapeutically relevant concentrations, and has further shown activity as an anti-inflammatory agent in an animal model of asthma. Unfortunately, the compound suffers from low bioavailability, and the acid form can exhibit low solubility.

Biologically active variants of the compounds set forth above are particularly also encompassed by the invention. Such variants should retain the general biological activity of the original compounds; however, the presence of additional activities would not necessarily limit the use thereof in the present invention. Such activity may be evaluated using standard testing methods and bioassays recognizable by the skilled artisan in the field as generally being useful for identifying such activity.

According to one embodiment of the invention, suitable biologically active variants comprise one or more analogues or derivatives of the compounds described above. Indeed, a single compound, such as those described above, may give rise to an entire family of analogues or derivatives having similar activity and, therefore, usefulness according to the present invention. Likewise, a single compound, such as those described above, may represent a single family member of a greater class of compounds useful according to the present invention. Accordingly, the present invention fully encompasses not only the compounds described above, but analogues and derivatives of such compounds, particularly those identifiable by methods commonly known in the art and recognizable to the skilled artisan.

The compounds disclosed herein may contain chiral centers, which may be either of the (R) or (S) configuration, or may comprise a mixture thereof. Accordingly, the present invention also includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the compounds described herein.

The compound of Formula (9), in particular, is a chiral compound, the chiral center being indicated with an asterisk. Accordingly, the antifolate compound of Formula (9) can exist as two separate enantiomers—either the (R) enantiomer or the (5) enantiomer. Typically, the antifolate compound of Formula (9) exists as a racemic mixture of the two enantiomers.

Various methods are known in the art for preparing optically active forms and determining activity. Such methods include standard tests described herein and other similar tests which are well known in the art. Examples of methods that can be used to obtain optical isomers of the compounds useful according to the present invention include the following:

i) physical separation of crystals whereby macroscopic crystals of the individual enantiomers are manually separated. This technique may particularly be used when crystals of the separate enantiomers exist (i.e., the material is a conglomerate), and the crystals are visually distinct;

ii) simultaneous crystallization whereby the individual enantiomers are separately crystallized from a solution of the racemate, possible only if the latter is a conglomerate in the solid state;

iii) enzymatic resolutions whereby partial or complete separation of a racemate by virtue of differing rates of reaction for the enantiomers with an enzyme;

iv) enzymatic asymmetric synthesis, a synthetic technique whereby at least one step of the synthesis uses an enzymatic reaction to obtain an enantiomerically pure or enriched synthetic precursor of the desired enantiomer;

v) chemical asymmetric synthesis whereby the desired enantiomer is synthesized from an achiral precursor under conditions that produce asymmetry (i.e., chirality) in the product, which may be achieved using chiral catalysts or chiral auxiliaries;

vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;

viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors whereby the desired enantiomer is obtained from non-chiral starting materials and where the stereochemical integrity is not or is only minimally compromised over the course of the synthesis;

x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography whereby the racemate is volatilized and enantiomers are separated by virtue of their differing interactions in the gaseous mobile phase with a column containing a fixed non-racemic chiral adsorbent phase;

xii) extraction with chiral solvents whereby the enantiomers are separated by virtue of preferential dissolution of one enantiomer into a particular chiral solvent; and

xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

In one embodiment, the pharmaceutical compositions of the invention comprise (S)-2-{4-[2-(2,4-diamino-quinazolin-6-yl)-ethyl]-benzoylamino}-4-methylene-pentanedioic acid, which is shown in Formula (10). The compound of Formula (10) is the (S) enantiomer of the compound shown in Formula (9). The (5) enantiomer is particularly useful in the pharmaceutical compositions of the invention in light of its increased activity in comparison to the (R) enantiomer.

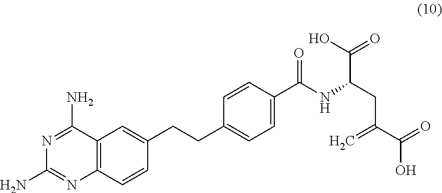

The antifolate compounds used in the inventive pharmaceutical compositions optionally may be provided in an enantiomerically enriched form, such as a mixture of enantiomers in which one enantiomer is present in excess (given as a mole fraction or a weight fraction). Enantiomeric excess is understood to exist where a chemical substance comprises two enantiomers of the same compound and one enantiomer is present in a greater amount than the other enantiomer. Unlike racemic mixtures, these mixtures will show a net optical rotation. With knowledge of the specific rotation of the mixture and the specific rotation of the pure enantiomer, the enantiomeric excess (abbreviated “ee”) can be determined by known methods. Direct determination of the quantities of each enantiomer present in the mixture (e.g., as a weight %) is possible with NMR spectroscopy and chiral column chromatography.

In one embodiment, the pharmaceutical compositions of the invention comprise a compound according to Formula (9), wherein the (S) enantiomer, as shown in Formula (10), is present in an enantiomeric excess. In such embodiments, the compositions can be referred to as comprising the compound of Formula (9) in an optically purified form in relation to the (S) enantiomer. Likewise, the compositions comprising an enantiomeric excess of the (S) enantiomer can be referred to as having a specific enantiomeric purity.

Preferably, the antifolate compounds used in the pharmaceutical compositions of the invention are enantiomerically pure for the (S) enantiomer such that greater than 50% of the compound present in the composition is the (S) enantiomer. In specific embodiments, the pharmaceutical compositions of the invention comprise an antifolate compound according to Formula (9) having an enantiomeric purity for the (S) enantiomer of at least about 75%. In other words, at least about 75% of the antifolate compound present in the composition is in the (S) form. In further embodiments, the antifolate compound of Formula (9) used in the inventive pharmaceutical compositions has an enantiomeric purity for the (S) enantiomer of at least about 80%, at least about 85%, at least about 90%, at least about 95%, at least about 96%, at least about 97%, at least about 98%, at least about 99%, at least about 99.5%, at least about 99.6%, at least about 99.7%, or at least about 99.8%.

The compounds described herein for use in the inventive pharmaceutical compositions can, in certain embodiments, be in the form of an ester, amide, salt, solvate, prodrug, or metabolite provided they maintain pharmacological activity according to the present invention. Esters, amides, salts, solvates, prodrugs, and other derivatives of the compounds of the present invention may be prepared according to methods generally known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

Examples of pharmaceutically acceptable salts of the compounds useful according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzesulfonic, and isethionic acids. Other useful acid addition salts include propionic acid, glycolic acid, oxalic acid, malic acid, malonic acid, benzoic acid, cinnamic acid, mandelic acid, salicylic acid, and the like. Particular example of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxyenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates. An acid addition salt may be reconverted to the free base by treatment with a suitable base.

If a compound of the invention is an acid, the desired salt may be prepared by any suitable method known to the art, including treatment of the free acid with an inorganic or organic base, such as an amine (primary, secondary or tertiary), an alkali metal or alkaline earth metal hydroxide or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum and lithium.

If a compound useful according to the invention is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such a p-toluenesulfonic acid or ethanesulfonic acid, or the like.

Esters of the compounds according to the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Moreover, esters and amides of compounds of the invention can be made by reaction with a carbonylating agent (e.g., ethyl formate, acetic anhydride, methoxyacetyl chloride, benzoyl chloride, methyl isocyanate, ethyl chloroformate, methanesulfonyl chloride) and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C. Prodrugs are typically prepared by covalent attachment of a moiety, which results in a compound that is therapeutically inactive until modified by an individual's metabolic system. Examples of pharmaceutically acceptable solvates include, but are not limited to, compounds according to the invention in combination with water, isopropanol, ethanol, methanol, DMSO, ethyl acetate, acetic acid, or ethanolamine.

In particular embodiments, the antifolate compound used in the pharmaceutical compositions comprises a salt of the antifolate compounds described above. In preferred embodiments, the invention provides a pharmaceutical composition comprising a salt of the compound according to Formula (11).

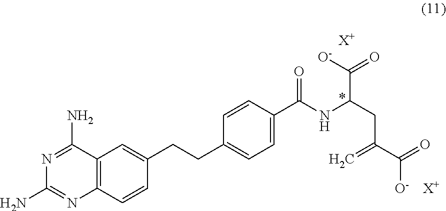

In Formula (11), the asterisk again denotes a chiral center, X⁺ can be any suitable salt-forming counterion, and each X⁺ can be the same or different. In specific embodiments, X⁺ is an alkali metal. In one preferred embodiment, X⁺ is a sodium cation. In another preferred embodiment, X⁺ is a potassium cation. In a specific embodiment, the composition of the invention comprises a disodium salt according to Formula (11). In still another specific embodiment, the composition of the invention comprises a dipotassium salt according to Formula (11). Of course, it is understood that other cationic moieties could be used as X⁺ in the compound of Formula (11). Moreover, the invention also encompasses salt forms according to Formula (11) that can be enantiomerically pure for the (R) enantiomer, enantiomerically pure for the (S) enantiomer, or in a racemic form. Such enantiomeric purity can be as previously described above.

Salts of antifolate compounds, such as the compounds of Formula (11), can be particularly useful in the pharmaceutical compositions of the invention in light of their favorable physico-chemical properties. The disodium salt of the compound of Formula (11) has particularly been shown to have improved solubility characteristics in comparison to the dioic acid form, as shown in Formula (9). This is further discussed in U.S. Patent Application Publication No. 20090253720, which is incorporated herein by reference in its entirety. This is particularly relevant in the case of pharmaceutical compositions of the invention for oral delivery, wherein the composition will encounter an acidic environment, such as in the stomach. Greater solubility of the sodium salt compound indicates a greater amount of the active compound will be available for absorption.

Although the invention clearly encompasses compositions comprising compounds in the salt form that are provided in a racemic mixture, in certain embodiments of the invention, it is particularly useful to provide pharmaceutical compositions comprising an antifolate compound that is in the salt form and that is enantiomerically purified for the (S) enantiomer. For example, in one embodiment, the invention provides a pharmaceutical composition comprising a disodium salt or a dipotassium salt of 2-{-4-[2-(2,4-diamino-quinazolin-6-yl)-ethyl]-benzoylamino}-4-methylene-pentanedioic acid that is enantiomerically purified for the (S) enantiomer, as described above. Accordingly, in a preferred embodiment, the invention provides a pharmaceutical composition comprising a compound according to Formula (12), which is a salt of (S)-2-{-4-[2-(2,4-diamino-quinazolin-6-yl)-ethyl]-benzoylamino}-4-methylene-pentanedioic acid, and wherein X⁺ is as defined above in relation to Formula (11). Preferably, the composition is at least 95% pure for the (5) enantiomer, more preferably at least 97% pure, still more preferably at least 98% pure, even more preferably at least 99% pure, and most preferably at least 99.5% pure for the (5) enantiomer.

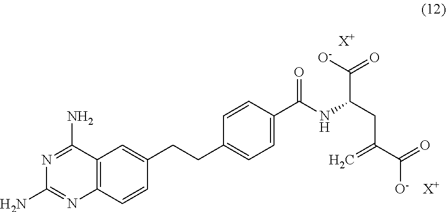

In the case of solid compositions, it is understood that the compounds used in the pharmaceutical compositions of the invention may exist in different forms. For example, the compounds may exist in stable and metastable crystalline forms and isotropic and amorphous forms, all of which are intended to be within the scope of the present invention.

Crystalline and amorphous forms of the inventive compounds can be characterized by the unique X-ray powder diffraction pattern (i.e., interplanar spacing peaks expressed in Angstroms) of the material. Equipment useful for measuring such data is known in the art, such as a Shimadzu XRD-6000 X-ray diffractometer, and any such equipment can be used to measure the compounds according to the present invention.

In specific embodiments, the invention comprises pharmaceutical compositions comprising antifolate compounds, as described above, in a stable crystalline form. In a specific embodiment, the pharmaceutical compositions comprise a salt compound according to Formula (11) in a stable crystalline form. In a preferred embodiment, the pharmaceutical compositions comprise a salt compound according to Formula (12) in a stable crystalline form, and wherein the compound has an enantiomeric purity for the (S) enantiomer as described herein.

In one embodiment of the invention, an antifolate compound used in the inventive compositions is a disodium salt characterized by the following approximate X-ray powder diffraction “d-spacing” peaks (i.e., interplanar spacing peaks at 2° θ): 4.8750, 7.3490, 8.1221, 10.5019, 11.8701, 12.4449, 14.5270, 16.0326, 17.1551, 20.6738, 21.1909, 21.7468, 22.5306, 23.2841, 23.9665, 24.4918, 28.3375, 29.1428, 30.8958, 32.2118, 33.5960, 34.5226, and 35.4153. Such salts are further characterized in U.S. Patent Application Publication No. 20090253719, which is incorporated herein by reference in its entirety.

The pharmaceutical compositions of the present invention further include prodrugs and active metabolites of the antifolate compounds of the invention. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. In preferred embodiments, the compounds of this invention possess anti-proliferative activity against abnormally proliferating cells, or are metabolized to a compound that exhibits such activity.

A number of prodrug ligands are known. In general, alkylation, acylation, or other lipophilic modification of one or more heteroatoms of the compound, such as a free amine or carboxylic acid residue, reduces polarity and allows passage into cells. Examples of substituent groups that can replace one or more hydrogen atoms on the free amine and/or carboxylic acid moiety include, but are not limited to, the following: aryl; steroids; carbohydrates (including sugars); 1,2-diacylglycerol; alcohols; acyl (including lower acyl); alkyl (including lower alkyl); sulfonate ester (including alkyl or arylalkyl sulfonyl, such as methanesulfonyl and benzyl, wherein the phenyl group is optionally substituted with one or more substituents as provided in the definition of an aryl given herein); optionally substituted arylsulfonyl; lipids (including phospholipids); phosphotidylcholine; phosphocholine; amino acid residues or derivatives; amino acid acyl residues or derivatives; peptides; cholesterols; or other pharmaceutically acceptable leaving groups which, when administered in vivo, provide the free amine and/or carboxylic acid moiety. Any of these can be used in combination with the disclosed compounds to achieve a desired effect.

Various processes for synthesizing antifolate compounds are disclosed in U.S. Pat. No. 4,996,207, U.S. Pat. No. 5,550,128, U.S. Pat. No. 5,912,251, Abraham et al. (1991) J. Med. Chem. 34:222-227, Rosowsky et al. (1991) J. Med. Chem. 34:203-208, and U.S. Patent Application Publication No. 20090253720, all of which are incorporated herein by reference.

### III. PHARMACEUTICAL COMPOSITIONS

The present invention particularly provides pharmaceutical compositions comprising one or more antifolate compounds as described herein or pharmaceutically acceptable esters, amides, salts, solvates, analogs, derivatives, or prodrugs thereof. Further, the inventive compositions can be prepared and delivered in a variety of combinations. For example, the composition can comprise a single composition containing all of the active ingredients. Alternately, the composition can comprise multiple compositions comprising separate active ingredients but intended to be administered simultaneously, in succession, or in otherwise close proximity of time.

The pharmaceutical compositions can be prepared to deliver one or more antifolate compounds together with one or more pharmaceutically acceptable excipients, and optionally, other therapeutic ingredients. Excipients should be acceptable in that they are compatible with any other ingredients of the composition and not harmful to the recipient thereof. An excipient may also reduce any undesirable side effects of the agent. Non-limiting examples of excipients that could be used according to the invention are described by Wang et al. (1980) J. Parent. Drug Assn. 34(6):452-462, herein incorporated by reference in its entirety.

In certain embodiments, the pharmaceutical compositions of the invention comprise one or more antifolate compounds, as described herein, in combination with one or more excipients. Surprisingly, choice of excipient(s) and methods of preparation of the pharmaceutical compositions can affect various properties of the compositions, including active content uniformity. Such effects were identified via rigorous, purposeful testing, and the present invention was able to identify appropriate compositions (and methods of preparation) that minimized or eliminated undesirable composition variability. Such testing is described in the Examples appended hereto.

As the antifolate compounds of the present invention typically can be provided in a solid form, one option for preparing a pharmaceutical composition is to simply dry blend the active compound with one or more excipients. It was discovered according to the present invention, however, that such blending methods proved unacceptable because of inconsistencies in the active content of individual dosage forms prepared by the dry blending method. See Example 2 below.

While not necessarily wishing to be bound by theory, one reason accounting for the unacceptable active content uniformity seen with simple dry blending of the antifolate compounds of the present invention is believed to be the varying particle size of the solid antifolates and the relatively large particle sizes included in the composition. Due to the nature of the compounds, the antifolates used in the present inventive compositions are not easily provided in a particulate form with consistent particle size. As seen in Example 2, dry blending using an active pharmaceutical ingredient (API) having a particle size ranging up to about 220 μm resulted in compositions wherein the group of individual dosages exhibited a percent relative standard deviation (% RSD) as high as 11.2%. This is particularly problematic since the antifolate compounds of the present invention may be used in relatively small dosages, for example as low as about 0.1 mg per dosage.

One option to increase standardization of the per-dosage content of the API (e.g., an antifolate compound as described herein, particularly a salt form of an antifolate compound described herein) is to mill or grind the API to a consistent, small particle size. Milling is a routine process for handling API powders in solid dosage forms and thus would be expected to be the route one of skill in the art would take for processing the API of the present invention for use in a pharmaceutical composition. A skilled person typically would be expected to use milling to achieve uniform particle size because it is recognized in the art that particle size is not controlled by milling at the end of a typical drug substance synthesis, and many drug substances for formulation are provided with non-uniform particle size. A skilled person also would understand that size reduction techniques usually ensure better mixing characteristics and content uniformity. Also, in most cases, milling is a fast, economical process for use in dosage form manufacturing.

The present inventors have recognized, however, that milling of antifolate compounds used in the present inventive compositions is undesirable. Specifically, the nature of the antifolate compounds makes milling a tedious and time consuming process that can be cost prohibitive. Moreover, milling of antifolate compounds useful according to the present invention can result in an unacceptable loss of API. Specific testing indicated that mechanical milling of API used according to the present invention was unsuccessful because of the nature of the API itself. In simple lab tests (i.e., using a mortar and pestle), applied mechanical force functioned to alter the nature of the API such that it partially transformed from a free-flowing state to an agglomerated state. On the lab scale, loss of API during milling was approximately 8-10% by weight. Scaling of the process only increased the API loss. For example, in batch sizes greater than 5 kg of API, a loss as high as 20% by weight was observed. In addition to the drug loss, milling was found to be ineffective for achieving uniform size reduction. As noted above, milling actually changed the physical nature of the compound such that the API powder became tacky or sticky, which led to reduced flow and cause particle agglomeration. While not wishing to be bound by theory, it is believed that such milling problems may arise from a somewhat hygroscopic nature of the present API. This further hindered successful milling in that process humidity had to be tightly controlled (e.g., less than 40% relative humidity) to ensure ambient moisture did not further reduce the API quality. Accordingly, such experimentation indicated that milling, although being an expected useful method, was in fact not useful and actually hindered achieving a successful pharmaceutical composition according to the present invention. The present invention, however, overcomes all of the aforementioned stumbling blocks and provides antifolate compositions with a highly consistent active content uniformity while still using an API in a particulate form with a relatively large particle size and a relatively large range of particle sizes.

In some embodiments, the inventive compositions thus can be described in terms of the size of the particles of the API used in the composition and/or used in the preparation of the composition. Such size can be particularly relevant since, as more fully described below, it is possible according to the invention to achieve a highly consistent active content uniformity in individual dosages of the inventive compositions without the need for milling of the API. For example, pharmaceutical compositions according to the present invention can comprise an antifolate compound, as described herein, that is in a solid, particulate form and wherein at least a portion of the particles have a size of at least about 50 μm, at least about 75 μm, at least about 100 μm, at least about 125 μm, at least about 150 μm, at least about 175 μm, or at least about 200 μm. In specific embodiments, at least about 5% by weight of the particles may have the above-noted particle size (e.g., at least about 5% by weight of the particles have a particle size of at least about 50 μm, at least about 5% by weight of the particles have a particle size of at least about 75 μm, etc.). In further embodiments, the above-noted particle sizes can apply to greater amounts of the particles, such as at least about 10% by weight, at least about 20% by weight, at least about 25% by weight, at least about 30% by weight, at least about 50% by weight, at least about 75% by weight, at least about 80% by weight, or at least about 90% by weight of the particles may have one of the above-noted particle sizes. Thus, the API may be described such that at least about 10%, 20%, 25%, 30%, 50%, 75%, 80%, or 90% by weight of the particles have a particle size of at least about 50 μm, 75 μm, 100 μm, 125 μm, 150 μm, 175 μm, or 200 μm.

In further embodiments, the particle size of the API can be described in relation to a size range. For example, in some embodiments, the API used in the inventive compositions can have a particle size in the range of about 10 μm to about 250 μm, about 20 μm to about 250 μm, about 30 μm to about 250 μm, about 40 μm to about 250 μm, about 50 μm to about 250 μm, about 60 μm to about 250 μm, about 70 μm to about 250 lam, about 80 μm to about 250 μm, about 90 μm to about 250 μm, about 100 μm to about 250 μm, about 110 μm to about 250 μm, about 120 μm to about 250 μm, about 130 μm to about 250 μm, about 140 μm to about 250 μm, about 150 μm to about 250 μm, about 160 μm to about 250 μm, about 170 μm to about 250 μm, about 180 μm to about 250 μm, about 190 μm to about 250 μm, or about 200 μm to about 250 μm. In further embodiments, a specific amount of the API particles may be within one of the noted ranges. For example, at least about 5% by weight of the particles may have a size range of about 150 μm to about 250 μm, at least about 5% by weight of the particles may have a size range of about 200 μm to about 250 μm, and so on. In further embodiments, the above-noted particle size ranges can apply to greater amounts of the particles, such as at least about 10% by weight, at least about 20% by weight, at least about 25% by weight, at least about 30% by weight, at least about 50% by weight, at least about 75% by weight, at least about 80% by weight, or at least about 90% by weight of the particles may fall within one of the above-noted particle size ranges. Thus, the API may be described such that at least about 10%, 20%, 25%, 30%, 50%, 75%, 80%, or 90% by weight of the particles may have a particle size in the range of about 10 μm to about 250 μm, about 20 μm to about 250 μm, about 30 μm to about 250 μm, about 40 μm to about 250 μm, about 50 μm to about 250 μm, about 60 μm to about 250 μm, about 70 μm to about 250 μm, about 80 μm to about 250 μm, about 90 μm to about 250 μm, about 100 μm to about 250 μm, about 110 μm to about 250 μm, about 120 μm to about 250 μm, about 130 μm to about 250 μm, about 140 μm to about 250 μm, about 150 μm to about 250 μm, about 160 μm to about 250 μm, about 170 μm to about 250 μm, about 180 μm to about 250 μm, about 190 μm to about 250 μm, or about 200 μm to about 250 μm.

The noted particle sizes, ranges, and contents within the sizes and ranges are particularly relevant in light of the ability to arrive at a highly consistent active content uniformity without the requirement of specific processing steps prior to formulation of the inventive compositions, such as costly and difficult milling of the API. In other words, API uniformity across dosage forms can be achieved while using particulate API having particles with sizes as described above, particularly wherein the particles span the entire range. Thus, the inventive compositions further can be described in terms of the actual active content uniformity of individual unit dosages of the pharmaceutical composition. Specifically, active content uniformity can be described in relation to the mean content of the API in the individual unit dosages in that the mean content between individual dosages varies by less than a specified amount. This specified amount can be expressed in a variety of ways, such as percent relative standard deviation. Although not intended to limit the scope of the invention herein, % RSD is used since it is a known statistical evaluation that one of skill in the art easily could use to evaluate whether a product exhibits an active content uniformity falling within the bounds of the present invention. Moreover, % RSD easily can be determined regardless of the desired mean content of the API. As recognized in probability theory and statistics, the relative standard deviation is the absolute value of the coefficient of variation. Relative standard deviation is widely used in analytical chemistry to express the precision and repeatability of an assay and could be calculated, for example, by the following equation: (standard deviation of array X)×100/(average of array X).

In specific embodiments, a pharmaceutical composition according to the present invention can be provided as individual unit dosages wherein the % RSD of the content of the API (e.g., the antifolate compound) in a group of the individual unit dosages is less than about 5%. In further embodiments, the % RSD of the API content in a group of the individual unit dosages may be less than about 4.5%, less than about 4%, less than about 3.5%, less than about 3%, less than about 2.5%, or less than about 2%. The group of individual unit dosages may be comprised of a single batch of dosages or a group of batches of dosages. In such embodiments, a “batch” may be defined to include the number of individual dosages typically prepared in commercial manufacturing of a drug product for dosing to an end user. Moreover, the group of individual dosages for evaluating % RSD may comprise about 5 to about 5,000, about 5 to about 2,500, about 5 to about 1,000, about 5 to about 500, about 10 to about 500, about 10 to about 250, or about 10 to about 100 individual dosages.

In preferred embodiments, the pharmaceutical composition of the present invention may be in a granular form. Thus, the inventive pharmaceutical compositions also may be described in relation to a specific granulation method used to prepare the composition. For example, in some embodiments, the inventive composition may be prepared by a wet granulation method. In particular, it has been found according to the present invention that, unlike when using a dry blending method, preparation using a wet granulation method as described herein allows for achieving the desired active content uniformity described herein without the need for additional processing steps, such as milling of the API prior to preparation of the composition.

In addition to the above, it has been found according to the present invention that specific granulation methods, such as wet granulation, also can be useful to ensure API content batch uniformity. Pharmaceutical compositions typically are formulated so that individual dosages will have a specific mean content. Ensuring uniformity of the mean content between individual dosage units is described above. It also is necessary, though, to ensure that each batch of the composition that is prepared is consistent such that the mean unit content is achieved, and this can be affected by the formulation method that is used. For example, as described in the Examples, several tested methods either were inconsistent in relation to mean API content between batches or simply were unable to achieve the desired mean content. It was found, however, that wet granulation not only achieved the desired % RSD, as described above, but also provided batch content uniformity such that the achieved API mean content in each prepared batch was within the desired range. Preferably, the method used provides batch content uniformity such that the mean API content in the unit dosages from each batch is within 5% of the desired API content for a unit dosage, preferably within 4%, within 3%, within 2%, within 1%, or within 0.5% of the desired API content. Such batch content uniformity also may be referred to in relation to the percent label strength of individual batches. For example, if the desired API dosage content is 1 mg (i.e., the label strength) and the mean per dosage API content for a given batch is 0.95 mg, the percent label strength for that batch would be 95%. Likewise, if the desired API dosage content is 1 mg (i.e., the label strength) and the mean per dosage API content for a given batch is 1.05 mg, the percent label strength for that batch would be 105%.

In preferred embodiments, the methods of the invention provide batch content uniformity such that the mean API content in the unit dosages from each batch exhibits a percent label strength (% LS) of at least about 90%, at least about 91%, at least about 92%, at least about 93%, at least about 94%, at least about 95%, at least about 96%, at least about 97%, at least about 98%, or at least about 99%. In further embodiments, the methods of the invention provide batch content uniformity such that the mean API content in the unit dosages from each batch exhibits a percent label strength of about 95% to about 105%, about 96% to about 104%, about 97% to about 103%, about 98% to about 102%, or about 99% to about 101%.

Specific wet granulation methods and compositions prepared according to such methods are provided in the Examples appended hereto. In some embodiments, a wet granulation method according to the present invention can comprise forming an intra-granular portion and blending the intra-granular portion with one or more extra-granular components. Preferably, the API is included in the intra-granular portion.

In one embodiment, wet granulation can comprise the following steps:
screening the dry components of the intra-granular portion except the API into the bowl of a granulator (such as a Diosna P1-6 Granulator);
mixing in the granulator;
dissolving the API in a portion of the liquid (e.g., water) used in the wet granulation;
adding the API solution to the dry components in the granulator
adding the remaining volume of liquid;
mixing to form the desired mixture;
transferring the mixture to a drying apparatus (e.g., a fluid bed dryer) and drying under specified conditions to reach a desired level of dryness;
passing the dried mixture through a granulator to form individual particles; and
blending the particles with the extra-granular components to form the final blend.

A wet granulation method useful for forming a pharmaceutical composition according to the present invention can be more particularly described as follows. The intra-granular components can be combined with the liquid component (which can be any pharmaceutically acceptable solvent, particularly water) and mixed to form a suitable granule. In specific embodiments, the granule is dried to a specified level of dryness. For example, the wet granule could be placed on a fluid bed dryer and dried at a suitable temperature. Preferably, heating is carried out for a time sufficient such that the loss on drying (LOD) reaches a desired range. Specifically, the desired LOD may be such that the final total liquid content of the granule (and thus the pharmaceutical composition) is about 3% (w/w) to about 8% (w/w), about 3.5% (w/w) to about 7.5% (w/w), about 4% (w/w) to about 7% (w/w), about 4% (w/w) to about 6.5% (w/w), or about 4.5% (w/w) to about 6% (w/w). It has been determined according to the present invention that maintaining a specified moisture range in the prepared granules is useful to provide good flow properties and further increase content uniformity. In specific embodiments, the combination of excipient choice with the defined moisture content is beneficial for good flow, granule homogeneity, and reduced weight variation in the dosage form. In specific embodiments, the API introduced to the wet granulation method can be characterized by the particle sizes described above.

As described herein, such properties particularly may be achieved through the use of specific combinations of excipients. For example, it has been found that the use of two specific types of microcrystalline cellulose as primary fillers can be beneficial for achieving such characteristics. The choice of the two different microcrystalline cellulose materials may be based on material particle size. For example, it can be useful to choose one type of microcrystalline cellulose having a first size range and a second type of microcrystalline cellulose having a second size range. The first material may have a size range of about 20 μm to about 125 μm, about 25 μm to about 100 μm, or about 30 μm to about 75 μm. The second material may have a size range of about 150 μm to about 250 μm, about 155 μm to about 225 μm, or about 160 μm to about 200 μm.

In a specific example according to one embodiment of the invention, microcrystalline cellulose products designated AVICEL® PH 101 and AVICEL® PH 200 may be used (although further products with similar physical characteristics could be used). AVICEL® PH 101 is particularly useful as an excipient in wet granulation methods, and AVICEL® PH 200 is particularly useful as an excipient in dry granulation methods. According to the present invention, however, it is useful to use both types of microcrystalline cellulose because the particle size of the AVICEL® PH 200 (i.e., an average of about 180 μm) was found to more closely match the average particle size of the further materials used in the inventive formulation. Moreover, it was found that the use of both types of microcrystalline cellulose as fillers in the formulation provided a synergistic effect in achieving better mixing and content uniformity. Still further, it was found that the use of both types of microcrystalline cellulose achieved a dual use formulation. Specifically, the inventive pharmaceutical composition can be provided in a capsule delivery form and, the composition can be successfully formed as a tablet dosage form. This is in part possible because the combination of fillers functions as a tableting aid as well as improving flow during mixing. Of course, other types of microcrystalline cellulose could be used alone or in combination according to the invention, and the choice of MCC fillers can depend upon the desired compaction characteristics, flow properties, granulation density, and granulation moisture content.

In some embodiments, it may be desirable to use only one type of microcrystalline cellulose product in combination with another type of filler, such as mannitol. Such combination according to the present invention is not intuitive since mannitol typically is used in dry granulation methods, not wet granulation. The use of mannitol (or other similar materials—e.g., other polyols) is particularly beneficial when used according to a specific method. For example, it can be useful to combine the microcrystalline cellulose and the mannitol into a single material, such as by co-spray drying. The combined product then can be added to the wet granulation method as otherwise described herein.

The intra-granular portion can comprise a number of different excipients, such as more fully described below. In particular embodiments, it has been found that the use of multiple binders, fillers, and compression aids in the intra-granular portion can be particularly useful in achieving the desired uniformity in individual unit dosages. In specific embodiments, it can be desirable to use one or more types of microcrystalline cellulose as such fillers (and particularly blends thereof) can provide for improved mixing, flow, and content uniformity. Other cellulose products may also be useful, such as carboxymethyl cellulose. One particularly useful cellulose product according to the invention is Ac-Di-Sol® (available from FMC Corporation), a cross-linked croscarmellose sodium carboxymethyl cellulose. In a specific embodiment, the intra-granular portion used in a wet-granulation method can include both mannitol and microcrystalline cellulose. In another specific embodiment, the intra-granular portion used in a wet-granulation method can include both AVICEL® PH200 and AVICEL®PH101 (both available from FMC Corporation). Other useful components include starch materials (e.g., STARCH 1500®, available from Colorcon, Inc.), polyols (such as mannitol—e.g., Mannitol SD200, also known as PEARLITOL® 200, available from Roquette Pharma), and binders (e.g., KOLLIDON® 30, available from BASF, AG, and hypromellose).

The extra-granular portion likewise can include a number of different excipients, such as described more fully below. In specific embodiments, the extra-granular portion can include a further amount of a binder, filler, or compression aid, such as a cellulose-based product (e.g., Ac-Di-Sol®). The extra-granular portion also can include one or more lubricants, such as fatty acids or salts thereof (e.g., magnesium stearate or stearic acid).

Examples of specific excipients that may be used in granulation methods according to the present invention include fillers/binders (such as cellulose materials or starch materials, e.g., pre-gelatinized starch), lubricants, wetting agents (such as sodium lauryl sulfate), disintegrants (such as sodium starch glycollate), and dissolution aids (such as cyclodextrins, which are further generally described in Comprehensive Supramolecular Chemistry, Volume 3, Cyclodextrins (Lehn, Jean-Marie and Osa, Tetsuo, editors), Elsevier Science, Inc., which is incorporated herein by reference in its entirety).

In specific embodiments, a composition according to the present invention prepared according to a granulation method described herein may comprise a specific content of various components. For example, such composition may comprise about 2% to about 98% by weight of at least one filler, such as cellulose-based or polyol-based materials. In other embodiments, such composition may comprise about 10% to about 98%, about 25% to about 95%, about 50% to about 95%, about 60% to about 95%, about 70% to about 95%, or about 80% to about 95% by weight of at least one filler. In preferred embodiments, two primary fillers (e.g., two different types of microcrystalline cellulose) may be used, each comprising about 5% to about 80% by weight of the total amount of primary filler present. More particularly, the two primary fillers each may comprise about 20% to about 70%, about 25% to about 65%, or about 30% to about 60% by weight of the total weight of the composition. Secondary fillers (e.g., STARCH 1500®) may comprise about 1% to about 15%, about 2.5% to about 12.5%, or about 5% to about 10% by weight of the total weight of the composition. Disintegrants (such as Ac-Di-Sol®) may comprise about 1% to about 10%, about 1% to about 7%, about 2% to about 7%, or about 3% to about 6% by weight of the total weight of the composition (said amount optionally being split by any ratio between the intra-granular portion and the extra-granular portion during preparation in the wet granulation method). Lubricants, such as magnesium stearate or stearic acid, may comprise about 0.1% to about 5%, about 0.1% to about 4%, about 0.1% to about 3%, about 0.1% to about 2%, about 0.2% to about 1.5%, or about 0.25% to about 1% by weight of the total weight of the composition. The API can be present in any amount as otherwise described herein.

Although the wet granulation method described above is a preferred method, in further embodiments, the desirable physical characteristics of the inventive composition can be achieved through further formulation methods. For example, the desired active content uniformity can be achieved by using a hot melt granulation method. Specific hot granulation methods and compositions prepared according to such methods are provided in the Examples appended hereto.

Unlike wet granulation, when using hot melt granulation, it can be beneficial to provide some degree of particle size reduction in relation to the API prior to the granulation. Preferably, the API particles used in a hot melt granulation method are sized so that at least about 95% by weight of the particles pass through a 70 mesh (i.e., 210 μm) screen, at least about 95% by weight of the particles pass through a 80 mesh (i.e., 180 μm) screen, at least about 95% by weight of the particles pass through a 100 mesh (i.e., 150 μm) screen, at least about 95% by weight of the particles pass through a 120 mesh (i.e., 125 μm) screen, at least about 95% by weight of the particles pass through a 140 mesh (i.e., 105 μm) screen, at least about 95% by weight of the particles pass through a 170 mesh (i.e., 90 μm) screen, or at least about 95% by weight of the particles pass through a 200 mesh (i.e., 75 μm) screen.

In one embodiment, hot melt granulation can comprise the following steps:
combining the API in a granulator bowl with a filler, such as microcrystalline cellulose, while heating;
adding a solubility/bioavailability enhancer (e.g., GELUCIRE® 44/14) and allowing it to fully liquefy;
mixing under high shear to achieve uniformity and form a suitable granule;
cooling the formed granule; and
screening the granule and a lubricant (e.g., magnesium stearate or stearic acid) into a container and blending the materials.

The holt melt process can be generally described as combining the API with a surface active excipient, preferentially a GELUCIRE® compound, and one or more further excipients. In further embodiments, other solubility/bioavailability enhancers could be used. Non-limiting examples of further solubility/bioavailability enhancers include tocopherol (i.e., vitamin-E), polyethyleneglycol compounds (e.g., PEG-4000), polyethylene glycol esters (e.g., LABRAFIL© 1944CS), polyvinylpyrrolidones (e.g., Povidone K29/32), polyethyleneoxide copolymers (e.g., LUTROL© F68), alkyl-pyrrolidones (e.g., PHARMASOLVE® and PHARMASOLVE®-Polysorbate 80), polyoxyethylene esters of fatty acids, such as polyoxyl esters of castor oil (e.g., CREMOPHOR© EL), sorbated vegetable oils (e.g., olive oil—Polysorbate 80), salts and esters of caprylic acid (e.g., CAPTEX® 355-Polysorbate 80 and ACCONON© MC8-2), and microcrystalline cellulose (e.g., AVICEL© PH 101).

GELUCIRE®, a product of Gattefosse s.a., Saint-Priest Cedex, France and Westwood, N.J., USA, is an excipient that is useful in various applications and is available in multiple forms having a range of properties. It is a semi-solid excipient formed of fatty acid esters of glycerol and polyethylene glycol esters (“PEG esters”) and can be described as a polyglycolized glyceride. Accordingly, these terms are also meant to be interchangeable as used herein and are meant to encompass GELUCIRE® compositions. Polyglycolized glycerides are inert semi-solid waxy materials which are amphiphilic in character and are available with varying physical characteristics. They are surface active in nature and disperse or solubilize in aqueous media forming micelles, microscopic globules, or vesicles. They are identified by their melting point/HLB value. The melting point is expressed in degrees Celsius and the HLB (Hydrophile-Lipophile Balance) is a numerical scale extending from 1 to approximately 20. Lower HLB values denote more lipophilic and hydrophobic substances, and higher values denote more hydrophilic and lipophobic substances. The affinity of a compound for water or for oily substances is determined and its HLB value is assigned experimentally. One or a mixture of different grades of polyglycolized glyceride excipient may be chosen to achieve the desired characteristics of melting point and/or HLB value. The appropriate choice of melting point/HLB value of a polyglycolized glyceride or a mixture of polyglycolized glyceride compositions will provide the delivery characteristics needed for a specific function, e.g., immediate release, sustained release, and the like.

In certain embodiments, it is preferable to use a polyglycolized glyceride compound having specific characteristics. For example, in specific embodiments, it is useful to choose a particular polyglycolized glyceride compound having a melting point that is less than about 50° C. In other embodiments, the polyglycolized glyceride can have a melting point in the range of about 33° C. to about 50° C. In further embodiments, the polyglycolized glyceride compound can be chosen based upon its HLB value. In specific embodiments, the polyglycolized glyceride compound has an HLB value that is greater than about 8. In other embodiments, the polyglycolized glyceride compound has an HLB value of about 8 to about 14. In even further embodiments, the polyglycolized glyceride can be chosen based upon the type of fatty acid or the type of PEG compound used. For example, it is useful for the fatty acid to be a glyceryl ester, such as glyceryl laurate, although any C₁₄-C₂₀ fatty acid ester could be used. In other embodiments, the PEG compound can be chosen based upon the molecular weight of the PEG compound (which is based on the total number of ethylene glycol groups present in the polymer). For example, the PEG compound can have a number average MW of about 1,200 to about 2,500 Da (i.e., PEG 1,000 to about PEG 2,000). In other embodiments, the PEG compound can range from about PEG 1200 to about PEG 1800, from about PEG 1300 to about PEG 1800, or from about PEG 1400 to about PEG 1600. GELUCIRE® 44/14 is particularly useful according to certain embodiments of the invention and is PEG1500 ester of glyceryl laurate having a melting point of 44° C. and an HLB of 14.

The amount of polyglycolized glyceride compound used in the pharmaceutical compositions of the invention can vary. In certain embodiments, the amount of polyglycolized glyceride compound is related to the amount of the antifolate compound used. For example, the weight-to-weight (w/w) ratio of polyglycolized glyceride to antifolate compound can be in the range of about 0.1:1 to about 80:1. In specific embodiments, the ratio of polyglycolized glyceride compound to antifolate compound is in the range of about 1:1 to about 50:1, about 2:1 to about 40:1, about 5:1 to about 25:1, or about 10:1 to about 20:1.

In other embodiments, the amount of polyglycolized glyceride compound used in the pharmaceutical formulations of the invention is based on the overall weight of the composition. For example, in certain embodiments, the pharmaceutical compositions of the invention comprise polyglycolized glyceride compound in an amount of up to about 250 mg per gram of overall composition. In further embodiments, the inventive pharmaceutical compositions comprise about 1 mg/g to about 250 mg/g, about 5 mg/g to about 200 mg/g, about 25 mg/g to about 175 mg/g, or about 50 mg/g to about 150 mg/g of polyglycolized glyceride compound, based on the weight of the overall pharmaceutical composition. The amount of the glyceride compound also may be referenced to the overall weight of the composition. For example, compositions according to the invention may comprise about 5% to about 50%, about 7.5% to about 45%, about 10% to about 40%, about 12.5% to about 35%, or about 15% to about 30% by weight of a glyceride compound, based on the overall weight of the composition.

The content of further materials in compositions made according to a hot melt granulation method may be as otherwise described herein. In some embodiments, the compositions formed by hot melt granulation may be described in relation to the ratio of filler to glyceride compound. For example, the w/w ratio of filler to glyceride compound may be about 90:10 to about 10:90, about 90:10 to about 50:50, about 88:12 to about 75:25, about 85:15 to about 78:22, or about 82:18 to about 80:20.

The pharmaceutical compositions of the invention preferably include an antifolate compound in a therapeutically effective amount, as further described below. In certain embodiments, the amount of antifolate compound in the compositions is based on the overall weight of the composition. For example, in certain embodiments, the pharmaceutical composition comprises an antifolate compound in an amount of about 0.01 mg/g to about 100 mg/g. In further embodiments, the pharmaceutical composition comprises an antifolate compound in an amount of about 0.02 mg/g to about 80 mg/g, about 0.05 mg/g to about 75 mg/g, about 0.08 mg/g to about 50 mg/g, about 0.1 mg/g to about 30 mg/g, about 0.25 mg/g to about 25 mg/g, or about 0.5 mg/g to about 20 mg/g. In other embodiments, the antifolate content may be referenced to a weight percent of the overall composition. In such embodiments, the compositions may comprise about 0.01% to about 20%, about 0.025% to about 15%, about 0.05 to about 12.5%, about 0.075% to about 10%, about 0.1% to about 10%, about 0.1% to about 7.5%, or about 0.1% to about 5% by weight of the composition. The amount of drug can also be referenced to a unit dose (e.g., the amount of drug in a single capsule or tablet). In specific embodiments, the compositions of the invention can provide an antifolate compound in an amount of about 0.1 mg to about 20 mg, about 0.1 mg to about 15 mg, about 0.1 mg to about 10 mg, about 0.2 mg to about 10 mg, about 0.2 mg to about 8 mg, or about 0.5 mg to about 5 mg. The content of the antifolate compound can be referenced to the content of the salt. In other embodiments, even when a salt form is used, the amount of the antifolate compound can be referenced to the content of the free acid present.

Compositions of the present invention may include short-term, rapid-onset, rapid-offset, controlled release, sustained release, delayed release, and pulsatile release compositions, providing the compositions achieve administration of a compound as described herein. See Remington's Pharmaceutical Sciences (18^{th} ed.; Mack Publishing Company, Eaton, Pa., 1990), herein incorporated by reference in its entirety.

Pharmaceutical compositions according to the present invention are suitable for various modes of delivery, including oral, parenteral (including intravenous, intramuscular, subcutaneous, intradermal, intra-articular, intra-synovial, intrathecal, intra-arterial, intracardiac, subcutaneous, intraorbital, intracapsular, intraspinal, intrasternal, and transdermal), topical (including dermal, buccal, and sublingual), pulmonary, vaginal, urethral, and rectal administration. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated. In preferred embodiments, the compositions of the present invention are provided in an oral dosage form, as more fully described below.

The pharmaceutical compositions may be conveniently made available in a unit dosage form, whereby such compositions may be prepared by any of the methods generally known in the pharmaceutical arts. Generally speaking, such methods of preparation comprise combining (by various methods) the active compounds of the invention with a suitable carrier or other excipient, which may consist of one or more ingredients. The combination of the active ingredients with the one or more adjuvants is then physically treated to present the composition in a suitable form for delivery (e.g., shaping into a tablet or forming an aqueous suspension).

Pharmaceutical compositions according to the present invention suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions according to the present invention.

In one embodiment, a compound according to the invention may be administered orally in combination with a pharmaceutically acceptable vehicle such as an inert diluent or an edible carrier or other excipient. Oral compositions may be enclosed in hard or soft shell gelatin capsules, may be compressed into tablets or may be incorporated directly with the food of the patient's diet. The percentage of the composition and preparations may be varied; however, the amount of substance in such therapeutically useful compositions is preferably such that an effective dosage level will be obtained.

Hard capsules containing the compound may be made using a physiologically degradable composition, such as gelatin. Such hard capsules comprise the compound, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the drug, various soluble excipients, such as lactose, powdered sucrose, dextrose, and mannitol. The solid dosage forms of the present invention may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT®), zein, shellac, and polysaccharides.

Powdered and granular compositions of a pharmaceutical preparation of the invention may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of the pharmaceutical composition of the invention which are suitable for oral administration may be prepared, packaged, and sold either in liquid form or in the form of a dry product intended for reconstitution with water or another suitable vehicle prior to use.

A tablet containing one or more compounds according to the present invention may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Adjuvants or accessory ingredients, in addition to those discussed above, for use in the compositions of the present invention can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluloses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the composition according to the present invention may be anionic, cationic, amphoteric, or nonionic surface active agents. Stabilizers may be included in the compositions to inhibit or lessen reactions leading to decomposition of the active agents, such as oxidative reactions.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agents over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

In certain embodiments, the compounds and compositions disclosed herein can be delivered via a medical device. Such delivery can generally be via any insertable or implantable medical device, including, but not limited to stents, catheters, balloon catheters, shunts, or coils. In one embodiment, the present invention provides medical devices, such as stents, the surface of which is coated with a compound or composition as described herein. The medical device of this invention can be used, for example, in any application for treating, preventing, or otherwise affecting the course of a disease or condition, such as those disclosed herein.

In another embodiment of the invention, the pharmaceutical compositions of the invention can be administered intermittently. Administration of the therapeutically effective dose may be achieved in a continuous manner, as for example with a sustained-release composition, or it may be achieved according to a desired daily dosage regimen, as for example with one, two, three, or more administrations per day. By “time period of discontinuance” is intended a discontinuing of the continuous sustained-released or daily administration of the composition. The time period of discontinuance may be longer or shorter than the period of continuous sustained-release or daily administration. During the time period of discontinuance, the level of the components of the composition in the relevant tissue is substantially below the maximum level obtained during the treatment. The preferred length of the discontinuance period depends on the concentration of the effective dose and the form of composition used. The discontinuance period can be at least 2 days, at least 4 days or at least 1 week. In other embodiments, the period of discontinuance is at least 1 month, 2 months, 3 months, 4 months or greater. When a sustained-release composition is used, the discontinuance period must be extended to account for the greater residence time of the composition in the body. Alternatively, the frequency of administration of the effective dose of the sustained-release composition can be decreased accordingly. An intermittent schedule of administration of a composition of the invention can continue until the desired therapeutic effect, and ultimately treatment of the disease or disorder, is achieved.

The inventive pharmaceutical compositions can comprise a single pharmaceutically active antifolate compound as described herein, can comprise two or more pharmaceutically active antifolate compounds as described herein, or can comprise one or more pharmaceutically active antifolate compounds as described herein with one or more further pharmaceutically active compounds (I.e., co-administration). Accordingly, it is recognized that the pharmaceutically active compounds in the compositions of the invention can be administered in a fixed combination (i.e., a single pharmaceutical composition that contains both active materials). Alternatively, the pharmaceutically active compounds may be administered simultaneously (i.e., separate compositions administered at the same time). In another embodiment, the pharmaceutically active compounds are administered sequentially (i.e., administration of one or more pharmaceutically active compounds followed by separate administration or one or more pharmaceutically active compounds). One of skill in the art will recognized that the most preferred method of administration will allow the desired therapeutic effect.

Delivery of a therapeutically effective amount of a composition according to the invention may be obtained via administration of a therapeutically effective dose of the composition. Accordingly, in one embodiment, a therapeutically effective amount is an amount effective to treat abnormal cell proliferation. In another embodiment, a therapeutically effective amount is an amount effective to treat inflammation. In yet another embodiment, a therapeutically effective amount is an amount effective to treat arthritis. In still another embodiment, a therapeutically effective amount is an amount effective to treat asthma.

The active compound is included in the pharmaceutical composition in an amount sufficient to deliver to a patient a therapeutic amount of a compound of the invention in vivo in the absence of serious toxic effects. The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time

A therapeutically effective amount according to the invention can be determined based on the bodyweight of the recipient. For example, in one embodiment, a therapeutically effective amount of one or more compounds of the invention is in the range of about 0.1 μg/kg of body weight to about 5 mg/kg of body weight per day. Alternatively, a therapeutically effective amount can be described in terms of a fixed dose. Therefore, in another embodiment, a therapeutically effective amount of one or more compounds of the invention is in the range of about 0.01 mg to about 500 mg per day. Of course, it is understood that such an amount could be divided into a number of smaller dosages administered throughout the day. The effective dosage range of pharmaceutically acceptable salts and prodrugs can be calculated based on the weight of the parent antifolate to be delivered. If a salt or prodrug exhibits activity in itself, the effective dosage can be estimated as above using the weight of the salt or prodrug, or by other means known to those skilled in the art.

It is contemplated that the compositions of the invention comprising one or more compounds described herein will be administered in therapeutically effective amounts to a mammal, preferably a human. An effective dose of a compound or composition for treatment of any of the conditions or diseases described herein can be readily determined by the use of conventional techniques and by observing results obtained under analogous circumstances. The effective amount of the compositions would be expected to vary according to the weight, sex, age, and medical history of the subject. Of course, other factors could also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

### IV. ACTIVE AGENT COMBINATIONS

For use in treating various diseases or conditions, the pharmaceutical compositions of the invention can include the antifolate compounds described above in various combinations. For example, in one embodiment, a pharmaceutical composition according to the invention can comprise a single antifolate compound described herein, such as the compound according to Formula (12). In another embodiment, a pharmaceutical composition according to the invention can comprise two or more antifolate compounds disclosed herein. In still further embodiments, a pharmaceutical composition according to the invention can comprise one or more antifolate compounds described herein with one or more further compounds known to have therapeutic properties. For example, the pharmaceutical compositions described herein can be administered with one or more toxicity-reducing compounds (e.g., folic acid or leucovorin). In further embodiments, the inventive pharmaceutical compositions can be administered with one or more compounds known to be an anti-inflammatory, anti-arthritic, antibiotic, antifungal, or antiviral agent. Such further compounds can be provided as a component of the pharmaceutical composition or can be provided in alternation with the compositions of the invention. In other words, the pharmaceutical compositions of the invention can be administered with the additional active agent(s) in the same composition with the antifolate compounds disclosed herein, or the additional active agent(s) can be administered in a separate delivery form from the pharmaceutical compositions of the invention. In particular embodiments, the pharmaceutical compositions of the invention can be provided in combination with one or more compounds selected from the groups described below.

In the following description, certain compounds useful as further active agents in the pharmaceutical compositions of the invention with the antifolate compounds disclosed above may be described in reference to specific diseases or conditions commonly treated using the noted compounds. The disclosure of such diseases or conditions is not intended to limit the scope of the invention and particularly does not limit the diseases or conditions that may be treated using the pharmaceutical compositions disclosed herein. Rather such exemplary diseases or conditions are provided only to illustrate the types of diseases and conditions typically treated using the additional compounds.

As additional active agents, the pharmaceutical compositions of the present invention can, in certain embodiments, be administered with antiproliferative agents. Proliferative disorders are currently treated by a variety of classes of compounds including alkylating agents, antimetabolites, natural products, enzymes, biological response modifiers, miscellaneous agents, radiopharmaceuticals (for example, Y-90 tagged to hormones or antibodies), hormones and antagonists. Any of the antiproliferative agents listed below or any other such therapeutic agents and principles as described in, for example, DeVita, V. T., Jr., Hellmann, S., Rosenberg, S. A.; Cancer: Principles & Practice of Oncology, 5th ed., Lippincott-Raven Publishers (1997), can be used with the pharmaceutical compositions of the present invention

Representative, nonlimiting examples of anti-angiogenesis agents suitable for use with the pharmaceutical compositions of the present invention include, but are not limited to, retinoid acid and derivatives thereof, 2-methoxyestradiol, ANGIOSTATINT™ protein, ENDOSTATINT™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs (1-azetidine-2-carboxylic acid (LACA), cis-hydroxyproline), d,1-3,4-dehydroproline, thiaproline, alpha,alpha-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chimp-3, chymostatin, beta-cyclodextrin tetradecasulfate, eponemycin, fumagillin, gold sodium thiomalate, d-penicillamine (CDPT), beta-1-anticollagenase-serum, alpha-2-antiplasmin, bisantrene, lobenzarit disodium, n-(2-carboxyphenyl-4-chloroanthronilic acid disodium or “CCA”, thalidomide, angostatic steroid, cargboxynaminolmidazole, and metalloproteinase inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

Representative, nonlimiting examples of alkylating agents suitable for use with the pharmaceutical compositions of the present invention include, but are not limited to, Nitrogen Mustards, such as Mechlorethamine (Hodgkin's disease, non-Hodgkin's lymphomas), Cyclophosphamide, Ifosfamide (acute and chronic lymphocytic leukemias, Hodgkin's disease, non-Hodgkin's lymphomas, multiple myeloma, neuroblastoma, breast, ovary, lung, Wilms' tumor, cervix, testis, soft-tissue sarcomas), Melphalan (L-sarcolysin) (multiple myeloma, breast, ovary), Chlorambucil (chronic lymphocytic leukemia, primary macroglobulinemia, Hodgkin's disease, non-Hodgkin's lymphomas), Ethylenimines and Methylmelamines, such as, Hexamethylmelamine (ovary), Thiotepa (bladder, breast, ovary), Alkyl Sulfonates, such as, Busulfan (chronic granulocytic leukemia), Nitrosoureas, such as, Carmustine (BCNU) (Hodgkin's disease, non-Hodgkin's lymphomas, primary brain tumors, multiple myeloma, malignant melanoma), Lomustine (CCNU) (Hodgkin's disease, non-Hodgkin's lymphomas, primary brain tumors, small-cell lung), Semustine (methyl-CCNU) (primary brain tumors, stomach, colon), Streptozocin (STR) (malignant pancreatic insulinoma, malignant carcinoin) and Triazenes, such as, Dacarbazine (DTIC—dimethyltriazenoimidazole-carboxamide) (malignant melanoma, Hodgkin's disease, soft-tissue sarcomas).

Representative, nonlimiting examples of anti-metabolite agents suitable for use with the pharmaceutical compositions of the present invention include, but are not limited to, Folic Acid Analogs, such as, Methotrexate (amethopterin) (acute lymphocytic leukemia, choriocarcinoma, mycosis fungoides, breast, head and neck, lung, osteogenic sarcoma), Pyrimidine Analogs, such as Fluorouracil (5-fluorouracil—5-FU) Floxuridine (fluorodeoxyuridine—FUdR) (breast, colon, stomach, pancreas, ovary, head and neck, urinary bladder, premalignant skin lesions) (topical), Cytarabine (cytosine arabinoside) (acute granulocytic and acute lymphocytic leukemias), Purine Analogs and Related Inhibitors, such as, Mercaptopurine (6-mercaptopurine-6-MP) (acute lymphocytic, acute granulocytic and chronic granulocytic leukemia), Thioguanine (6-thioguanine-TG) (acute granulocytic, acute lymphocytic and chronic granulocytic leukemia), Pentostatin (2′-deoxycyoformycin) (hairy cell leukemia, mycosis fungoides, chronic lymphocytic leukemia), Vinca Alkaloids, such as, Vinblastine (VLB) (Hodgkin's disease, non-Hodgkin's lymphomas, breast, testis), Vincristine (acute lymphocytic leukemia, neuroblastoma, Wilms' tumor, rhabdomyosarcoma, Hodgkin's disease, non-Hodgkin's lymphomas, small-cell lung), Epipodophylotoxins, such as Etoposide (testis, small-cell lung and other lung, breast, Hodgkin's disease, non-Hodgkin's lymphomas, acute granulocytic leukemia, Kaposi's sarcoma), and Teniposide (testis, small-cell lung and other lung, breast, Hodgkin's disease, non-Hodgkin's lymphomas, acute granulocytic leukemia, Kaposi's sarcoma).

Representative, nonlimiting examples of cytotoxic agents suitable for use with the pharmaceutical compositions of the present invention include, but are not limited to: doxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci.

Non-limiting examples of natural products suitable for use with the inventive pharmaceutical compositions include, but are not limited to: Antibiotics, such as, Dactinomycin (actinonmycin D) (choriocarcinoma, Wilms' tumor rhabdomyosarcoma, testis, Kaposi's sarcoma), Daunorubicin (daunomycin-rubidomycin) (acute granulocytic and acute lymphocytic leukemias), Doxorubicin (soft tissue, osteogenic, and other sarcomas, Hodgkin's disease, non-Hodgkin's lymphomas, acute leukemias, breast, genitourinary thyroid, lung, stomach, neuroblastoma), Bleomycin (testis, head and neck, skin and esophagus lung, and genitourinary tract, Hodgkin's disease, non-Hodgkin's lymphomas), Plicamycin (mithramycin) (testis, malignant hypercalcemia), Mitomycin (mitomycin C) (stomach, cervix, colon, breast, pancreas, bladder, head and neck), Enzymes, such as, L-Asparaginase (acute lymphocytic leukemia), and Biological Response Modifiers, such as, Interferon-alpha (hairy cell leukemia, Kaposi's sarcoma, melanoma, carcinoid, renal cell, ovary, bladder, non Hodgkin's lymphomas, mycosis fungoides, multiple myeloma, chronic granulocytic leukemia).

Additional agents that can be used with the inventive compositions include, but are not limited to: Platinum Coordination Complexes, such as, Cisplatin (cis-DDP) Carboplatin (testis, ovary, bladder, head and neck, lung, thyroid, cervix, endometrium, neuroblastoma, osteogenic sarcoma); Anthracenedione, such as Mixtozantrone (acute granulocytic leukemia, breast); Substituted Urea, such as, Hydroxyurea (chronic granulocytic leukemia, polycythemia vera, essential thrombocytosis, malignant melanoma); Methylhydrazine Derivatives, such as, Procarbazine (N-methylhydrazine, MIH) (Hodgkin's disease); Adrenocortical Suppressants, such as, Mitotane (o,p′-DDD) (adrenal cortex), Aminoglutethimide (breast); Adrenorticosteriods, such as, Prednisone (acute and chronic lymphocytic leukemias, non-Hodgkin's lymphomas, Hodgkin's disease, breast); Progestins, such as, Hydroxprogesterone caproate, Medroxyprogesterone acetate, Megestrol acetate (endometrium, breast); and Steroids, such as betamethasone sodium phosphate and betamethasone acetate.

Representative, nonlimiting examples of hormones and antagonists suitable for use with the pharmaceutical compositions of the present invention include, but are not limited to, Estrogens: Diethylstibestrol Ethinyl estradiol (breast, prostate); Antiestrogen: Tamoxifen (breast); Androgens: Testosterone propionate Fluxomyesterone (breast); Antiandrogen: Flutamide (prostate); Gonadotropin-Releasing Hormone Analog: and Leuprolide (prostate). Other hormones include medroxyprogesterone acetate, estradiol, megestrol acetate, octreotide acetate, diethylstilbestrol diphosphate, testolactone, and goserelin acetate.

The pharmaceutical compositions of the present invention can be used with therapeutic agents used to treat arthritis. Examples of such agents include, but are not limited to, the following:

Nonsteroidal anti-inflammatory drugs (NSAIDs), such as cylcooxygenase-2 (COX-2) inhibitors, aspirin (acetylsalicylic acid), ibuprofen, ketoprofen, naproxen, and acetaminophen;

Analgesics, such as acetaminophen, opioid analgesics, and transdermal fentanyl;

Biological response modifiers, such as etanercept, infliximab, adalimumab, anakinra, abatacept, tiruximab, certolizumab pegol, and tocilizumab;

Corticosteroids or steroids, such as glucocorticoids (GC), fluticasone, budesonide, prednisolone, hydrocortisone, adrenaline, Aldosterone, Cortisone Acetate, Desoxymethasone, Dexamethasone, Fluocortolone, Hydrocortisone, Meprednisone, Methylprednisolone, Prednisolone, Prednisone, Prednylidene, Procinonide, Rimexolone, and Suprarenal Cortex;

Disease-modifying antirheumatic drugs (DMARDs), such as hydroxychloroquine, cyclosphosphamide, chlorambucil, the gold compound auranofin, sulfasalazine, minocycline, cyclosporine, toll-like receptor agonists and antagonists, kinase inhibitors (e.g., p38 MAPK) immunosuppressants and tumor necrosis factor (TNF) blockers (e.g., etanercept, infliximab, and adalimumab);

Fibromyalgia medications, such as amitriptyline, fluoxetine, cylobenzaprine, tramadol, gabapentin, pregabalin, and dual-reuptake inhibitors;

Osteoporosis medications, such as estrogens, parathyroid hormones, bisphosphonates, selective receptor molecules, and bone formation agents;

Gout medications, such as allopurinol, probenecid, losartan, and fenofibrate;

Psoriasis medications, such as acitretin; and

Topical treatments, such as topical NSAIDs and capsaicin.

The pharmaceutical compositions of the present invention also can be used with therapeutic agents used to treat asthma. Examples of such agents include, but are not limited to, the following:

Anti-allergics, such as cromolyn sodium and ketotifen fumarate;

Anti-inflammatories, such as NSAIDs and steroidal anti-inflammatories (e.g., beclomethasone dipropionate, budesonide, dexamethasone sodium phosphate, flunisolide, fluticasone propionate, and triamcinolone acetonide);

Anticholinergics, such as ipratropium bromide, belladonna alkaloids, atropine, and oxitropium bromide;

Antihistamines, such as chlorpheniramine, brompheniramine, diphenhydramine, clemastine, dimenhydrinate, cetirizine, hydroxyzine, meclizine, fexofenadine, loratadine, and enadine;

β₂-adrenergic agonists (beta agonists), such as albutamol, terbutaline, epinephrine, metaproterenol, ipratropium bromide, ephedra (source of alkaloids), ephedrine, and psuedoephedrine;

Leukotriene Receptor Antagonists, such as zafirlukast and zileuton montelukast;

Xanthines (bronchodilators), such as theophylline, dyphylline, and oxtriphylline;

Miscellaneous anti-asthma agents, such as xanthines, methylxanthines, oxitriphylline, aminophylline, phosphodiesterase inhibitors such as zardaverine, calcium antagonists such as nifedipine, and potassium activators such as cromakalim; and

Prophylactic agent(s), such as sodium cromoglycate, cromolyn sodium, nedocromil, and ketotifen.

Further, non-limiting examples of active agents that can be used with the pharmaceutical compositions of the present invention include anti-psoriasis agents, anti-Inflammatory Bowel Disease (anti-IBD) agents, anti-chronic obstructive pulmonary disease (anti-COPD) agents, anti-multiple sclerosis agents.

### V. ARTICLES OF MANUFACTURE

The present invention also includes an article of manufacture providing a pharmaceutical compositions comprising one or more antifolate compounds disclosed herein, optionally in combination with one or more further active agents. The article of manufacture can include a vial or other container that contains a composition suitable for use according to the present invention together with any excipient, either dried or in liquid form. In particular, the article of manufacture can comprise a kit including a container with a composition according to the invention. In such a kit, the composition can be delivered in a variety of combinations. For example, the composition can comprise a single dosage comprising all of the active ingredients. Alternately, where more than one active ingredient is provided, the composition can comprise multiple dosages, each comprising one or more active ingredients, the dosages being intended for administration in combination, in succession, or in other close proximity of time, including administration within a defined time span (e.g., within the same day or 24 hour period, within the same week of 7 day period, or within the same month or 30 day period). For example, the dosages could be solid forms (e.g., tablets, caplets, capsules, or the like) or liquid forms (e.g., vials), each comprising a single active ingredient, but being provided in blister packs, bags, or the like, for administration in combination or within a defined time span, as described above. For example, a one day kit could provide one, two, three, or more doses of a first composition and only a single dose of a second composition. Likewise, a one week kit could provide enough doses of a first composition for one, two, three, or more administrations per day and provide enough doses of a second composition for lesser administrations, such as only one or two administrations per week. Similarly, a one month kit could provide enough doses of a first composition for one, two, three, or more administrations per day and provide enough doses of a second composition for lesser administrations, such as only one or two administrations per week or one or two administrations per month.

The article of manufacture further includes instructions in the form of a label on the container and/or an insert included in a box in which the container is packaged, for the carrying out the inventive method. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

### VI. METHODS OF TREATMENT

As previously noted, antifolates can vary as to the folate-dependant metabolic process inhibited thereby, and many antifolates act on a variety of enzymes. Pemetrexed (also known as ALIMTA® or L-glutamic acid, N-[4-[2-(2-amino-4,7-dihydro-4-oxo-1H-pyrrolo[2,3-d]pyrimidin-5-yl)ethyl]benzoyl-, disodium salt, heptahydrate) is one example of an antifolate known to act on multiple enzymes. In particular, pemetrexed is known to exhibit antineoplastic activity by inhibiting TS, DHFR, and GARFT.

Thymidylate synthase (TS) is a rate-limiting enzyme in pyrimidine de novo deoxynucleotide biosynthesis and is therefore often a target for chemotherapeutic strategies. In DNA synthesis, TS plays a central role in reductive methylation of deoxyuridine-5′-monophosphate (dUMP) to deoxythymidine-5′-monophosphate (dTMP). Thus, TS inhibition leads directly to depletion of dTMP and subsequently of 2′-deoxythymidine-5′-triphosphate (dTTP), an essential precursor for DNA. This indirectly results in an accumulation of 2′-deoxyuridine-5′-triphosphate (dUTP) and, therefore, leads to so-called “thymine-less death” due to misincorporation of dUTP into DNA and subsequent excision catalyzed by uracil-DNA glycosylase, which causes DNA damage. Both this DNA damage and the noted imbalance in dTTP/dUTP can induce downstream events, leading to apoptosis (cell death).

Dihydrofolate reductase (DHFR) catalyzes the NADPH-dependent reduction of 7,8-dihydrofolate (DHF or H2F) to 5,6,7,8-tetrahydrofolate (THF or H4F). Thus, DHFR is necessary for maintaining intracellular levels of THF, an essential cofactor in the synthetic pathway of purines, thymidylate, and several amino acids.

Glycinamide ribonucleotide formyltransferase (GARFT) is a folate-dependent enzyme in the de novo purine biosynthesis pathway critical to cell division and proliferation. Specifically, GARFT catalyzes the formation of purines from the reaction of 10-formyltetrahydrofolate (10-FTHF) to THF Inhibition of GARFT results in a depletion in intracellular purine levels, which in turn inhibits DNA and RNA synthesis. Ultimately, disruption of DNA and RNA synthesis by GARFT inhibition results in cell death. The antiproliferative effect associated with GARFT inhibition makes it a particularly desirable target for anti-tumor drugs.

Antifolates, such as pemetrexed, can be transported into cells by mechanisms such as the reduced folate carrier system and the membrane folate binding protein transport system. Once in the cell, pemetrexed is converted to polyglutamylate forms by folyl polyglutamate synthase. The polyglutamylate forms are retained in cells and are inhibitors of TS and GARFT. Polyglutamylation is a time- and concentration-dependent process that occurs in tumor cells and, to a lesser extent, in normal tissues. Polyglutamylated metabolites have an increased intracellular half-life resulting in prolonged drug action in malignant cells.

In many instances, broad action against multiple enzymes may not be desirable. For example, pemetrexed inhibits DHFR, TS, and GARFT. As described above, inhibition of TS and GARFT is strongly related to cell death, thus the desirability of using TS and GARFT inhibitors as anti-tumor drugs. However, the ability of drugs, such as pemetrexed, to induce apoptosis increases the toxicity of the drug (i.e., death of healthy cells as well as tumor cells).

The function of compounds, such as pemetrexed, as inhibitors of TS and GARFT arises from the polyglutamylation of the compound inside the cell. Accordingly, compounds that are non-polyglutamylatable would not be expected to function as a TS inhibitor or a GARFT inhibitor. However, inhibition of polyglutamylation does not generally affect the ability of a compound to function as a DHFR inhibitor. For example, pemetrexed has been shown to have equivalent DHFR inhibition in comparison to the polyglutamate forms of pemetrexed.

The antifolate compounds used in the pharmaceutical compositions of the invention comprise a 4-methylidene group in the glutamate moiety of the compounds. Such may also be referred to as a gamma methylene glutamate moiety. The presence of the methylene group makes the antifolate compounds non-polyglutamylatable. Accordingly, the compounds of the invention are specific for DHFR inhibition (i.e., the compounds do not inhibit TS or GARFT due to the absence of polyglutamylation inside cells). Such specificity is desirable to provide for more specific treatments while avoiding or reducing toxicity and minimizing side-effects more commonly associated with compounds, such as pemetrexed, which act on additional enzymes, such as TS and GARFT.

The antifolate compounds used in the pharmaceutical compositions of the present invention are particularly useful in the treatment of various conditions wherein disruption of folic acid metabolism is beneficial for treating a symptom of the condition or the condition generally. Accordingly, in further embodiments, the present invention can be directed to methods of treating various diseases or conditions. In particular embodiments, the invention can provide methods of treating diseases or conditions known or found to be treatable by disruption of folic acid metabolism. In specific embodiments, the invention provides methods of treating conditions, such as abnormal cell proliferation, inflammation, arthritis, disorders of the cardiovascular system, and asthma. In further embodiments, the invention provides methods of treating rheumatoid arthritis, juvenile arthritis, psoriatic arthritis, psoriasis, diseases of the eye (including uveitis), inflammatory bowel disease, lupus, multiple sclerosis, leukemia, lymphoma, solid tumors, coronary heart disease, and atherosclerosis.

A. Abnormal Cellular Proliferation

Abnormal cell proliferation has been shown to be the root of many diseases and conditions, including cancer and non-cancer disorders which present a serious health threat. Generally, the growth of the abnormal cells, such as in a tumor, exceeds and is uncoordinated with that of normal cells. Furthermore, the abnormal growth of tumor cells generally persists in an abnormal (i.e., excessive) manner after the cessation of stimuli that originally caused the abnormality in the growth of the cells. A benign tumor is characterized by cells that retain their differentiated features and do not divide in a completely uncontrolled manner. A benign tumor is usually localized and nonmetastatic. A malignant tumor (i.e., cancer) is characterized by cells that are undifferentiated, do not respond to the body's growth control signals, and multiply in an uncontrolled manner. Malignant tumors are invasive and capable of metastasis.

Treatment of diseases or conditions of abnormal cellular proliferation comprises methods of killing, inhibiting, or slowing the growth or increase in size of a body or population of abnormally proliferative cells (including tumors or cancerous growths), reducing the number of cells in the population of abnormally proliferative cells, or preventing the spread of abnormally proliferative cells to other anatomic sites, as well as reducing the size of a growth of abnormally proliferative cells. The term “treatment” does not necessarily mean to imply a cure or a complete abolition of the disorder of abnormal cell proliferation. Prevention of abnormal cellular proliferation comprises methods which slow, delay, control, or decrease the likelihood of the incidence or onset of disorders of abnormal cell proliferation, in comparison to that which would occur in the absence of treatment.

Abnormal cellular proliferation, notably hyperproliferation, can occur as a result of a wide variety of factors, including genetic mutation, infection, exposure to toxins, autoimmune disorders, and benign or malignant tumor induction. Hyperproliferative cell disorders include, but are not limited to, skin disorders, blood vessel disorders, cardiovascular disorders, fibrotic disorders, mesangial disorders, autoimmune disorders, graft-versus-host rejection, tumors, and cancers.

Representative, non-limiting types of non-neoplastic abnormal cellular proliferation disorders that can be treated using the present invention include: skin disorders such as psoriasis, eczerma, keratosis, basal cell carcinoma, and squamous cell carcinoma; disorders of the cardiovascular system such as hypertension and vasculo-occlusive diseases (e.g., atherosclerosis, thrombosis and restenosis); blood vessel proliferative disorders such as vasculogenic (formation) and angiogenic (spreading) disorders which result in abnormal proliferation of blood vessels, such as antiogenesis; and disorders associated with the endocrine system such as insulin resistant states including obesity and diabetes mellitus (types 1 & 2).

The inventive compositions and methods are also useful for treating inflammatory diseases associated with non-neoplastic abnormal cell proliferation including, for example, inflammatory bowel disease (IBD), rheumatoid arthritis (RA), multiple sclerosis (MS), proliferative glomerulonephritis, lupus erythematosus, scleroderma, temporal arteritis, thromboangiitis obliterans, mucocutaneous lymph node syndrome, asthma, host versus graft, thyroiditis, Grave's disease, antigen-induced airway hyperactivity, pulmonary eosinophilia, Guillain-Barre syndrome, allergic rhinitis, myasthenia gravis, human T-lymphotrophic virus type 1-associated myelopathy, herpes simplex encephalitis, inflammatory myopathies, atherosclerosis, and Goodpasture's syndrome.

In a particular embodiment, the pharmaceutical compositions of the present invention are useful in the treatment of psoriasis. Psoriasis is an immune-mediated skin disorder characterized by chronic T-cell stimulation by antigen-presenting cells (APC) occurs in the skin. The various types of psoriasis include, for example, plaque psoriasis (i.e., vulgaris psoriasis), pustular psoriasis, guttate psoriasis, inverse psoriasis, erythrodermic psoriasis, psoriatic arthritis, scalp psoriasis and nail psoriasis. Common systemic treatments for psoriasis include methotrexate, cyclosporin and oral retinoids, but their use is limited by toxicity. Up to 40% of patients with psoriasis also develop psoriatic arthritis (Kormeili T et al. Br J. Dermatol. (2004) 151(0:3-15.

In further embodiments, the pharmaceutical compositions of the present invention are useful in the treatment of blood vessel proliferative disorders, including vasculogenic (formation) and angiogenic (spreading) disorders which result in abnormal proliferation of blood vessels. Other blood vessel proliferative disorders include arthritis and ocular diseases such as diabetic retinopathy. Abnormal neovascularization is also associated with solid tumors. In a particular embodiment, the compositions of the present invention are useful in the treatment of diseases associated with uncontrolled angiogenesis. Representative, non-limiting diseases of abnormal angiogenesis include rheumatoid arthritis, ischemic-reperfusion related brain edema and injury, cortical ischemia, ovarian hyperplasia and hypervascularity, (polycystic ovary syndrome), endometriosis, psoriasis, diabetic retinopathy, and other ocular angiogenic diseases such as retinopathy of prematurity (retrolental fibroplastic), macular degeneration, corneal graft rejection, neuroscular glaucoma, and Oster Webber syndrome. Cancers associated with abnormal blood cell proliferation include hemangioendotheliomas, hemangiomas, and Kaposi's sarcoma.

In further embodiments, the pharmaceutical compositions of the present invention are useful in the treatment of disorders of the cardiovascular system involving abnormal cell proliferation. Such disorders include, for example, hypertension, vasculo-occlusive diseases (e.g., atherosclerosis, thrombosis, and restenosis after angioplasty), acute coronary syndromes (such as unstable angina, myocardial infarction, ischemic and non-ischemic cardiomyopathies, post-MI cardiomyopathy, and myocardial fibrosis), and substance-induced cardiomyopathy.

Vascular injury can also result in endothelial and vascular smooth muscle cell proliferation. The injury can be caused by traumatic events or interventions (e.g., angioplasty, vascular graft, anastomosis, organ transplant) (Clowes A et al. A. J. Vasc. Surg (1991) 13:885). Restenosis (e.g., coronary, carotid, and cerebral lesions) is the main complication of successful balloon angioplasty of the coronary arteries. It is believed to be caused by the release of growth factors as a result of mechanical injury to the endothelial cells lining the coronary arteries.

Other atherosclerotic conditions which can be treated or prevented by means of the present invention include diseases of the arterial walls that involve proliferation of endothelial and/or vascular smooth muscle cells, including complications of diabetes, diabetic glomerulosclerosis, and diabetic retinopathy.

In further embodiments, the pharmaceutical compositions of the present invention are useful in the treatment of abnormal cell proliferation disorders associated the endocrine system. Such disorders include, for example, insulin resistant states including obesity, diabetes mellitus (types 1 & 2), diabetic retinopathy, macular degeneration associated with diabetes, gestational diabetes, impaired glucose tolerance, polycystic ovarian syndrome, osteoporosis, osteopenia, and accelerated aging of tissues and organs including Werner's syndrome.

In further embodiments, the pharmaceutical compositions of the present invention are useful in the treatment of abnormal cell proliferation disorders of the urogenital system. These include, for example, edometriosis, benign prostatic hyperplasia, eiomyoma, polycystic kidney disease, and diabetic nephropathy.

In further embodiments, the inventive pharmaceutical compositions are useful in the treatment of fibrotic disorders. Medical conditions involving fibrosis include undesirable tissue adhesion resulting from surgery or injury. Non-limiting examples of fibrotic disorders include hepatic cirrhosis and mesangial proliferative cell disorders.

In still further embodiments, abnormal cell proliferation disorders of the tissues and joints can be treated according to the present invention. Such disorders include, for example, Raynaud's phenomenon/disease, Sjogren's Syndrome systemic sclerosis, systemic lupus erythematosus, vasculitides, ankylosing spondylitis, osteoarthritis, reactive arthritis, psoriatic arthritis, and fibromyalgia.

In certain embodiments, abnormal cell proliferation disorders of the pulmonary system can also be treated according to the present invention. These disorders include, for example, asthma, chronic obstructive pulmonary disease (COPD), reactive airway disease, pulmonary fibrosis, and pulmonary hypertension.

Further disorders including an abnormal cellular proliferative component that can be treated according to the invention include Behcet's syndrome, fibrocystic breast disease, fibroadenoma, chronic fatigue syndrome, acute respiratory distress syndrome (ARDS), ischemic heart disease, post-dialysis syndrome, leukemia, acquired immune deficiency syndrome, vasculitis, lipid histiocytosis, septic shock, and familial intestinal polyposes such as Gardner syndrome. Other exemplary conditions that also are included in the scope of disorders that may be treated by the compounds, compositions, and methods of the present invention are virus-induced hyperproliferative diseases including, for example, human papilloma virus-induced disease (e.g., lesions caused by human papilloma virus infection), Epstein-Barr virus-induced disease, scar formation, genital warts, cutaneous warts, and the like.

The pharmaceutical compositions of the present invention are further useful in the treatment of conditions and diseases of abnormal cell proliferation including various types of cancers such as primary tumors and tumor metastasis. Specific, non-limiting types of benign tumors that can be treated according to the present invention include hemangiomas, hepatocellular adenoma, cavernous hemangiomas, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, and pyogenic granulomas. Other types of benign tumors also would be encompassed by the compounds, compositions, and methods of the present invention.

Representative, non-limiting examples of types of cancers that can be treated according to the invention include breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, reticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas.

The pharmaceutical compositions of the present invention are also useful in preventing or treating proliferative responses associated with organ transplantation which contribute to rejections or other complications. For example, proliferative responses may occur during transplantation of the heart, lung, liver, kidney, and other body organs or organ systems.

B. Inflammation

The pharmaceutical compositions of the present invention also can be useful in the treatment of diseases that are characterized by inflammation. Diseases and conditions which have significant inflammatory components are ubiquitous and include, for example, skin disorders, bowel disorders, certain degenerative neurological disorders, arthritis, autoimmune diseases and a variety of other illnesses. Some of these diseases have both an inflammatory and proliferative component, as also described above. In particular embodiments the compounds of the invention can be used to treat inflammatory bowel diseases (IBD), Crohn's disease (CD), ulcerative colitis (UC), chronic obstructive pulmonary disease (COPD), sarcoidosis, or psoriasis. The disclosed pharmaceutical compositions are also useful in the treatment of other inflammatory diseases, for example, allergic disorders, skin disorders, transplant rejection, poststreptococcal and autoimmune renal failure, septic shock, systemic inflammatory response syndrome (SIRS), adult respiratory distress syndrome (ARDS), envenomation, lupus erythematosus, Hashimoto's thyroiditis, autoimmune hemolytic anemias, insulin dependent diabetes mellitus, and rheumatic fever, pelvic inflammatory disease (PID), conjunctivitis, dermatitis, and bronchitis.

Inflammatory bowel diseases (IBD) includes several chronic inflammatory conditions, including Crohn's disease (CD) and ulcerative colitis (UC). Both CD and UC are considered “idiopathic” because their etiology is unknown. While Crohn's disease and ulcerative colitis share many symptoms (e.g., diarrhea, abdominal pain, fever, fatigue), ulcerative colitis is limited to the colon whereas Crohn's disease can involve any segment of the gastrointestinal tract. Both diseases may involve extraintestinal manifestations, including arthritis, diseases of the eye (e.g., episcleritis and iritis), skin diseases (e.g., erythema nodosum and pyoderma gangrenosum), urinary complications, gallstones, and anemia. Strokes, retinal thrombi, and pulmonary emboli are not uncommon, because many patients are in a hypercoagulable state.

In a particular embodiment, the pharmaceutical compositions of the present invention are useful in the treatment of inflammatory bowel disease. In a preferred embodiment, the inflammatory bowel disease is Crohn's disease.

The invention also can encompass treatment of Chronic Obstructive Pulmonary Disease, or COPD, which is characterized by a not fully reversible airflow limitation which is progressive and associated with an abnormal inflammatory reaction of the lungs. It is one of the most common respiratory conditions of adults, a major cause of chronic morbidity and mortality, and represents a substantial economic and social burden worldwide (Pauwels R A. Lancet. (2004) 364(9434):613-20). Other names for the disorder include, for example, Chronic Obstructive Airways Disease, (COAD); Chronic Obstructive Lung Disease, (COLD), Chronic Airflow Limitation, (CAL or CAFL) and Chronic Airflow Obstruction (COA).

COPD is characterized by chronic inflammation throughout the airways, parenchyma, and pulmonary vasculature. The inflammation involves a multitude of cells, mediators, and inflammatory effects. Mediators include, for example, mediators include proteases, oxidants and toxic peptides. Over time, inflammation damages the lungs and leads to the pathologic changes characteristic of COPD. Manifestations of disease includes both chronic bronchitis and emphysema. Chronic bronchitis is a long-standing inflammation of the airways that produces a lot of mucus, causing wheezing and infections. It is considered chronic if a subject has coughing and mucus on a regular basis for at least three months a year and for two years in a row. Emphysema is a disease that destroys the alveolae and/or bronchae, causing the air sacs to become enlarged, thus making breathing difficult. Most common in COPD patients is the centrilobular form of emphysema. In a particular embodiment, the compositions of the present invention are useful in the treatment of chronic obstructive pulmonary disease.

Sarcoidosis is yet another chronic inflammatory disease with associated abnormal cell proliferation. Sarcoidois is a multisystem granulomatous disorder wherein the granulomas are created by the angiogenic capillary sprouts providing a constant supply of inflammatory cells.

As noted above, inflammation also plays an important role in the pathogenesis of cardiovascular diseases, including restenosis, atherosclerotic complications resulting from plaque rupture, severe tissue ischemia, and heart failure. Inflammatory changes in the arterial wall, for example, are thought to play a major role in the development of restenosis and atherosclerosis (Ross R. N Engl J. Med. (1999) 340: 115-126). Local inflammation occurs in the formation the plaques also contributes to the weakening of the fibrous cap of the advanced plaque, ultimately resulting in plaque rupture and acute coronary syndromes (Lind L. Atherosclerosis. (2003) 169(2):203-14).

Multiple sclerosis (MS) is a chronic, often debilitating autoimmune disease that affects the central nervous system. MS is characterized by inflammation which results when the body directs antibodies and white blood cells against proteins in the myelin sheath, fatty material which insulates the nerves in the brain and spinal cord. The result may be multiple areas of scarring (sclerosis), which slows or blocks muscle coordination, visual sensation and other nerve signals. In a particular embodiment, the pharmaceutical compositions of the present invention can be useful in the treatment of multiple sclerosis and related conditions.

Some inflammatory conditions have been shown to be associated with the pathogenesis of neurological disorders, including Parkinson's disease and Alzheimer's disease (Mirza B. et al. Neuroscience (2000) 95(2):425-32; Gupta A. Int J Clin Pract. (2003) 57(1):36-9; Ghatan E. et al. Neurosci Biobehav Rev. (1999) 23(5):615-33). In specific embodiments, the present invention further encompasses treatment of such neurological disorders.

The present invention also can be useful in the treatment of further conditions. For example, the invention encompasses allergic disorders, allergic rhinitis, skin disorders, transplant rejection, poststreptococcal and autoimmune renal failure, septic shock, systemic inflammatory response syndrome (SIRS), adult respiratory distress syndrome (ARDS), envenomation, lupus erythematosus, myasthenia gravis, Grave's disease, Hashimoto's thyroiditis, autoimmune hemolytic anemias, insulin dependent diabetes mellitus, glomerulonephritis, and rheumatic fever, pelvic inflammatory disease (PID), conjunctivitis, dermatitis, bronchitis, and rhinitis.

C. Asthma

In particular embodiments the pharmaceutical compositions can be used in the treatment of asthma. In recent years, it has become clear that the primary underlying pathology of asthma is airway tissue inflammation (Lemanke (2002) Pediatrics 109(2):368-372; Nagayama et al. (1995) Pediatr Allergy Immunol. 6:204-208). Asthma is associated with numerous symptoms and signs (e.g., wheezing, cough, chest tightness, shortness of breath and sputum production). Airway inflammation is a key feature of asthma pathogenesis and its clinical manifestations. Inflammatory cells, including mast cells, eosinophils, and lymphocytes, are present even in the airways of young patients with mild asthma.

Inflammation also plays a role in wheezing disorders, with or without asthma. Asthma is sometimes classified by the triggers that may cause an asthma episode (or asthma attack) or the things that make asthma worse in certain individuals, such as occupational asthma, exercise induced asthma, nocturnal asthma, or steroid resistant asthma. Thus, the pharmaceutical compositions of the invention can also be used in the treatment of wheezing disorders, generally.

D. Arthritis and Osteoarthritis

More than 40 million Americans suffer from arthritic conditions. The term arthritis can include over 100 kinds of rheumatic diseases (i.e., diseases affecting joints, muscle, and connective tissue, which makes up or supports various structures of the body, including tendons, cartilage, blood vessels, and internal organs). Representative types of arthritis include rheumatoid (such as soft-tissue rheumatism and non-articular rheumatism), fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, spondyloarthropaties (ankylosing spondylitis), osteoarthritis, hyperuricemia and arthritis associated with acute gout, chronic gout, and systemic lupus erythematosus.

Hypertrophic arthritis or osteoarthritis is the most common form of arthritis and is characterized by the breakdown of the joint's cartilage. Osteoarthritis is common in people over 65, but may appear decades earlier. Breakdown of the cartilage causes bones to rub against each other, causing pain and loss of movement. In recent years, there has been increasing evidence that inflammation plays an important role in osteoarthritis. Nearly one-third of patients ready to undergo joint replacement surgery for osteoarthritis (OA) had severe inflammation in the synovial fluid that surrounds and protects the joints. In a particular embodiment, the pharmaceutical compositions of the present invention are useful in the treatment of osteoarthritis.

The second most common form of arthritis is rheumatoid arthritis. It is an autoimmune disease that can affect the whole body, causing weakness, fatigue, loss of appetite, and muscle pain. Typically, the age of onset is much earlier than osteoarthritis, between ages 20 and 50. Inflammation begins in the synovial lining and can spread to the entire joint. In another embodiment, the pharmaceutical compositions of the present invention are useful in the treatment of rheumatoid arthritis.

### EXPERIMENTAL

The present invention now is described with reference to various examples. The following examples are not intended to be limiting of the invention and are rather provided as exemplary embodiments. As used in one or more examples below, “CH-4051” refers to a compound of formula (12) that is purified for the L-form (i.e., the S enantiomer), wherein X⁺ is either sodium or potassium.

### Example 1

### Comparative Dry Blend Formulations and Method

Three formulations at a batch size of 50 g were manufactured to determine the active content uniformity using a dry blending method. The three initial formulations investigated are shown below in Table 1. For each formulation shown in Table 1, the method of manufacture for dry blending was as follows. Item 1 was screened through a 250 μm screen along with approximately 20% of Item 2, and the components were mixed together for approximately 2 minutes to form a pre-blend. This pre-blend was then screened through a 250 μm screen along with the remaining amount of Item 2, and the resulting mixture was blended together for approximately 2 minutes to form a second pre-blend. This second pre-blend was screened through a 250 μm screen along with Items 3, 4, and 5 (where applicable) and blended together for approximately 2 minutes to form a third pre-blend. Item 6 was then screened through a 250 μm screen into the third pre-blend, and this mixture was blended together for approximately 2 minutes to form the final dry blend.

TABLE 1

| | | Batch 1 | | Batch 2 | | Batch 3 | |
|---|---|---|---|---|---|---|---|
| Item | Material | mg/cap | % w/w | mg/cap | % w/w | mg/cap | % w/w |
| 1 | CH-4051 | 0.344 | 0.172 | 0.344 | 0.712 | 0.344 | 0.172 |
| 2 | STARCH 1500 ® | 100.000 | 50 | 100.000 | 50 | 100.000 | 50 |
| 3 | Mannitol SD200 | 88.656 | 44.328 | NA | NA | 70.000 | 35 |
| 4 | AVICEL ® PH200 | NA | NA | 88.656 | 44.328 | 18.656 | 9.328 |
| 5 | Ac-Di-Sol ® | 10.000 | 5 | 10.000 | 5 | 10.000 | 5 |
| 6 | Magnesium stearate | 1.000 | 0.5 | 1.000 | 0.5 | 1.000 | 0.5 |
| | Total: | 200.00 | 100 | 200.000 | 100 | 200.000 | 100 |

Ten samples of each batch were subjected to analytical testing to determine the API content batch uniformity in terms of the comparative achieved mean content in relation to the target content. Testing also was done to determine the active content uniformity between individual dosages (% RSD). The API mean target was 0.172%, and the test results are provided below in Table 2.

TABLE 2

| Batch No. | Mean Content (% w/w) | % RSD |
|---|---|---|
| 1 | 0.171 | 10.3 |
| 2 | 0.172 | 4.3 |
| 3 | 0.132 | 7.6 |

As seen above, API batch content uniformity was good in relation to batches 1 and 2, but batch 3 provided very poor results. Further, batches 1 and 3 exhibited poor active content uniformity between individual dosage forms. Batch 2 was evaluated to have provided the best results overall; however, in light of the variable results across all formulations, it was decided to repeat the manufacture of batch 2. Batch 4 thus was manufactured exactly as described above using the exact formulation as used for batch 2. The analytical results of batch 4 are provided below in Table 3.

TABLE 3

| Batch No. | Mean Content (% w/w) | % RSD |
|---|---|---|
| 4 | 0.171 | 11.2 |

As seen in Table 3, although the mean content of batch 4 was acceptable, the % RSD indicated very poor active content uniformity between individual dosage forms. To further verify the difficulties in achieving an acceptable formulation using dry blending it was decided to manufacture a fifth batch using a slightly different formulation (i.e., that did not include STARCH 1500®), since it was questioned whether the starch was leading to filtration blockages. This fifth formulation is shown below in Table 4, and the resulting uniformity test results are provided thereafter in Table 5.

TABLE 4

Batch 5

| Item | Material | mg/cap | % w/w |
|---|---|---|---|
| 1 | CH-4051 | 0.344 | 0.172 |
| 2 | AVICEL ® PH200 | 68.656 | 34.328 |
| 3 | Mannitol SD200 | 120.00 | 60 |
| 4 | Ac-Di-Sol ® | 10.000 | 5 |
| 5 | Magnesium stearate | 1.000 | 0.5 |
| | Total: | 200.00 | 100 |

TABLE 5

| Batch No. | Mean Content (% w/w) | % RSD |
|---|---|---|
| 5 | 0.155 | 10.3 |

As seen in Table 5, the testing of batch 5 verified the inconsistency in API content batch uniformity and further exemplified the inability to achieve an acceptable % RSD. Microscopic evaluation of the API indicated a particle size of up to about 220 μm. Given this relatively large particle size and the low dose mean content, it was determined that dry blending was not an acceptable method for preparation of compositions that would provide a consistently acceptable API content batch uniformity and active content uniformity between individual dosages (% RSD).

### Example 2

### Inventive Wet Granulation Formulations and Method

An initial formulation (batch 6) at a batch size of 50 g was manufactured to determine the active content uniformity using a wet granulation method. The analytical results for this batch showed that the wet granulation gave a % RSD of approximately 2.3. A larger batch size then was manufactured to confirm the initial results. The confirmatory formulation tested is shown below in Table 6.

TABLE 6

Batch 6

| | | | |
|---|---|---|---|
| Item | Material | mg/cap | % w/w |

Intra Granular Portion

| | | | |
|---|---|---|---|
| 1 | CH-4051 | 0.344 | 0.172 |
| 2 | Mannitol SD200 | 100.00 | 50 |
| 3 | AVICEL ® PH101 | 73.565 | 36.828 |
| 4 | STARCH 1500 ® | 15.000 | 7.5 |
| 5 | Ac-Di-Sol ® | 5.000 | 2.5 |
| 6 | Sterile Water | NA | NA |

Extra Granular Portion

| | | | |
|---|---|---|---|
| 7 | Ac-Di-Sol ® | 5.000 | 2.5 |
| 8 | Magnesium stearate | 1.000 | 0.5 |
| | Total: | 200.00 | 100 |

The wet granulation method used to manufacture the composition described above was as follows. Items 2, 3, 4, and 5 were passed through a 1.0 mm screen into a high speed granulator bowl and mixed for two minutes using the main impellor to form an intra granular portion pre-mix. Item 1 was dissolved in approximately 10% of the total water required for the 0.5 mg granulation to form an API aqueous solution. The API aqueous solution was added to the pre-mix in the granulator with the main impellor and granulator operating. This was followed by the remaining quantity of water. The material was mixed until a suitable granule was formed. The granule was transferred to a fluid bed dryer and dried using an inlet temperature of 60° C. until the LOD reached 4.5%-6.0% (w/w) (using LOD balance at 105° C. for 15 minutes). The dried granule was passed through a reciprocating dry granulator fitted with a 1.0 mm screen. The resultant granule was blended with items 7 and 8 (i.e., the extra granular portion). To make a dosage form, the final blend was filled into size 0 hard gelatin capsules using a dosating pin/funnel encapsulation machine and size 1 pin/funnel.

Ten samples of the final blend (identified as batch 7) were subjected to analytical testing to determine the API content batch uniformity in terms of the comparative achieved mean content in relation to the target content. Testing also was done to determine the active content uniformity between individual dosages (% RSD). The API mean target was 0.172% w/w, and the test results are provided below in Table 7. As seen below, the formulation of batch 7 prepared by the wet granulation method was shown to have excellent uniformity.

TABLE 7

| Batch No. | Mean Content (% w/w) | % RSD |
|---|---|---|
| 7 | 0.174 | 1.6 |

In light of the excellent results achieved using the wet granulation method, two further formulations were prepared at different API strengths. The formulations are provided below in Table 8. The formulations from Batches 8 and 9 were transferred to a Macofar encapsulation machine and approximately 2,000 capsules of each dose were auto filled to a target fill weight of 200 mg. The encapsulation machine was fitted with size 0 change parts and a size 1 dosating pin for the filling of granules.

TABLE 8

| | | Batch API Unit Dose: | | | |
|---|---|---|---|---|---|
| | | Batch 8 | | Batch 9 | |
| | | (0.5 mg API) | | (5.0 mg API) | |
| Item | Material | mg/cap | % w/w | mg/cap | % w/w |

Intra Granular Portion

| | | | | | |
|---|---|---|---|---|---|
| 1 | CH4051 | 0.688* | 0.344 | 6.88 | 3.44 |
| 2 | AVICEL ® PH200 | 100.00 | 50.0 | 100.00 | 50.0 |
| 3 | AVICEL ® PH101 | 73.312** | 36.656 | 67.12** | 33.56 |
| 4 | STARCH 1500 ® | 15.00 | 7.5 | 15.00 | 7.5 |
| 5 | Ac-Di-Sol ® | 5.00 | 2.5 | 5.00 | 2.5 |
| 6 | Sterile Water | qs*** | qs*** | qs*** | qs*** |

Extra Granular Portion

| | | | | | |
|---|---|---|---|---|---|
| 7 | Ac-Di-Sol ® | 5.00 | 2.5 | 5.00 | 2.5 |
| 8 | Magnesium Stearate | 1.00 | 0.5 | 1.00 | 0.5 |

*API free base content was based upon 100 mg of API containing 72.67 mg of free base after correction for purity, water, and potassium contents.
**AVICEL ® PH200 content adjusted after API calculation performed to maintain capsule fill weight of 200.0 mg.
***Removed during drying (total quantity of was required was approximately 50% by weight of items 1-5 inclusive)

The capsules prepared as described above were subjected to stability testing after packaging into 60 ml HDPE bottles that were induction sealed. Specifically, the capsules inside the bottles were stored under two different condition sets and periodically evaluated to determine the percent label strength (% LS) of the API in the capsules. Results after 6 months of testing are provided below in Table 9.

TABLE 9

| | | Batch 8 | | Batch 9 | |
|---|---|---|---|---|---|
| | | (0.5 mg API) | | (5.0 mg API) | |
| | | % Label | | % Label | |
| Time | | Strength (% LS) | | Strength (% LS) | |
| (months) | Conditions | Individual | Mean | Individual | Mean |
| 0 | NA | 100.570 | 101.6 | 100.046 | 101.4 |
| | | 103.738 | | 101.839 | |
| | | 100.442 | | 102.237 | |
| 1 | 25° C./60% RH | 95.383 | 96.3 | 96.835 | 97.6 |
| | | 97.263 | | 98.329 | |
| | 40° C./75% RH | 95.626 | 96.4 | 98.034 | 96.7 |
| | | 97.076 | | 95.410 | |
| 3 | 25° C./60% RH | 96.649 | 97.1 | 98.495 | 98.2 |
| | | 97.650 | | 97.939 | |
| | 40° C./75% RH | 97.422 | 97.2 | 95.352 | 96.3 |
| | | 96.902 | | 97.278 | |
| 6 | 25° C./60% RH | 96.908 | 97.7 | 99.824 | 99.0 |
| | | 98.507 | | 98.177 | |
| | 40° C./75% RH | 95.210 | 95.4 | 99.310 | 98.6 |
| | | 95.575 | | 97.892 | |

### Example 3

### Comparative High Shear Mixing Method

As a comparative to the wet granulation method, a high shear mixing method was carried out on the formulation described below in Table 10. CAVAMAX® W7 is a β-cyclodextrin product available from Wacker Chemie AG. All remaining components are as otherwise described herein. The general method of manufacture for the high shear mixing was a follows. Item 1 was ground and screened through an 80 μm screen. Item 1 and a 25% portion of Item 2 were blended together for 2 minutes to form a pre-blend. The pre-blend, the remainder of Item 2, and all of Items 3-5 were screened through a 1 mm screen and transferred to the bowl of a granulator where they were mixed for 2 minutes using the mixing blade only. All materials then were mixed for 5 minutes using both the mixing and granulating blades at high speed. The material was transferred to a suitable container, Item 6 was screened through a 1 mm screen into the container, and the materials were blended together for 2 minutes.

TABLE 10

Batch 10

| Item | Material | mg/cap | % w/w |
|---|---|---|---|
| 1 | CH-4051 | 0.344 | 0.172 |
| 2 | AVICEL ® PH200 | 100.00 | 50 |
| 3 | Mannitol SD200 | 70.00 | 35 |
| 4 | CAVAMAX ® W7 | 18.656 | 9.328 |
| 5 | Ac-Di-Sol ® | 10.000 | 5 |
| 6 | Magnesium stearate | 1.000 | 0.5 |
| | Total: | 200.00 | 100 |

The mean target API amount for the above formulation was 0.172% w/w, and the analytical results of batch 10 are provided below in Table 11. The high shear mixing method was determined to be unsatisfactory in light of the poor API content batch uniformity (i.e., low mean content) and the % RSD value being higher than that achieved using the wet granulation method.

TABLE 11

| Batch No. | Mean Content (% w/w) | % RSD |
|---|---|---|
| 10 | 0.160 | 3.2 |

### Example 4

### Inventive Hot Melt Granulation Formulations and Method

A formulation prepared by hot melt granulation (Batch 11) is shown below in Table 12. The general method of manufacture for the hot melt granulation method was as follows. Item 1 was ground and screened through an 80 μm screen. Item 1 and a portion of Item 2 were then blended together for approximately 2 minutes to form a pre-blend. The granulating bowl of a Diosna P1-6 granulator was heated to approximately 55° C. The pre-blend and the remainder of Item 2 were screened through a 500 μm screen and transferred to the granulating bowl. The materials were blended together using the impellor only for approximately 5 minutes to raise the temperature of the materials to approximately 42° C. All of Item 3 was added to the heated mixture, and all materials were blended together for approximately one minute using the impellor only. This mixture was left resting in the bowl for approximately eight minutes to allow the GELUCIRE® to melt fully. This mixture was then granulated with high shear mixing using the impellor and granulator until visual assessment showed that a satisfactory granule had been made. The granule was then removed from the bowl and placed into a stainless steel tray and allowed to cool for approximately 30 minutes. The cooled granule was screened through a 1.4 mm screen, Item 4 was screened through a 500 μm screen into the granule, and the materials were blended together for approximately two minutes.

TABLE 12

Batch 11

| Item | Material | mg/cap | % w/w |
|---|---|---|---|
| 1 | CH-4051 | 0.344 | 0.172 |
| 2 | AVICEL ® PH102 | 158.656 | 79.328 |
| 3 | GELUCIRE ® 44/14 | 40.00 | 20 |
| 4 | Magnesium stearate | 1.000 | 0.5 |
| | Total: | 200.00 | 100 |

The analytical results of batch 11 are provided below in Table 13. As seen below, the mean content was below the target of 0.172% w/w, but the % RSD value was very good. Test evaluation indicated that some amount of the API may have been lost during manufacturing.

TABLE 13

| Batch No. | Mean Content (% w/w) | % RSD |
|---|---|---|
| 11 | 0.159 | 1.9 |

In light of the good results achieved using the hot melt granulation method, a further formulation (Batch 12) was prepared at a different API strength. The formulation is provided below in Table 14.

TABLE 14

Batch 12

| Item | Material | mg/cap | % w/w |
|---|---|---|---|
| 1 | CH-4051 | 0.688 | 0.344 |
| 2 | AVICEL ® PH102 | 158.312 | 79.156 |
| 3 | GELUCIRE ® 44/14 | 40.00 | 20 |
| 4 | Magnesium stearate | 1.000 | 0.5 |
| | Total: | 200.00 | 100 |

The same method of manufacture was followed with the exception that a 180 μm screen was used initially to screen the API. The resulting product was transferred to a Macofar encapsulation machine and approximately 2,000 capsules of each dose were auto filled to a target fill weight of 200 mg. The encapsulation machine was fitted with size 0 change parts and a size 1 dosating pin for the filling of granules. The weight uniformity of the capsules filling was shown to be very good.

### Example 5

### Testing of Formulations Prepared by Wet Granulation and Hot Melt Granulation

Additional analytical testing on the inventive formulations of Batches 8, 9, 11, and 12 prepared according to inventive wet granulation (WG) and hot melt granulation (HMG) methods were carried out, and the results are provided below in Table 15. To carry out the tests, capsules prepared by each method were randomly selected and analyzed. An assay was performed on a representative number of capsules to determine an average for the initial percent label strength (% LS), or purity, of the formulations. To determine active content uniformity between individual dosages, 10 capsules from each formulation were evaluated, and the % RSD was determined along with % LS. Further, a dissolution test was carried out to determine the amount of drug released from the capsules as a function of time (reported as % LS and % RSD).

TABLE 15

| | WG Batch | | WG Batch 9 | | HMG Batch 11 | | HMG Batch 12 | |
|---|---|---|---|---|---|---|---|---|
| Formulation: | (0.5 mg API) | | (5.0 mg API) | | (0.5 mg API) | | (0.5 mg API) | |
| | % LS | % RSD | % LS | % RSD | % LS | % RSD | % LS | % RSD |
| Content Uniformity | | | | | | | | |
| Mean of 10 | 97.9 | 2.0 | 98.6 | 1.8 | 96.7 | 2.9 | 94.9 | 1.7 |
| Drug Release on | | | | | | | | |
| Dissolution (time) | | | | | | | | |
| 15 minutes | 92.8 | 3.9 | 97.1 | 3.4 | 88.7 | 11.1 | 80.8 | 7.0 |
| 30 minutes | 94.0 | 2.7 | 98.7 | 1.6 | 90.9 | 8.2 | 84.2 | 5.5 |
| 45 minutes | 93.9 | 2.4 | 98.4 | 1.5 | 91.6 | 6.8 | 85.7 | 3.9 |
| 60 minutes | 93.6 | 2.2 | 97.6 | 1.4 | 91.7 | 6.0 | 86.2 | 2.9 |

| Assay | % LS | % LS | % LS | % LS |
|---|---|---|---|---|
| | 97.8 | 99.8 | 98.5 | 93.3 |

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A pharmaceutical composition comprising: about 0.01% to about 20% by weight of an antifolate compound according to Formula (6): 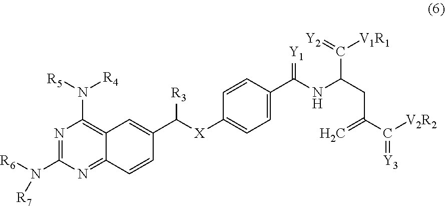
wherein:
X is CHR₈ or NR₈;
Y₁, Y₂, and Y₃ independently are O or S;
V₁ and V₂ independently are O, S, or NZ;
Z is H, optionally substituted alkyl, optionally substituted alkenyl, optionally substituted alkynyl, or alkaryl;
R₁ and R₂ independently are H, optionally substituted alkyl, optionally substituted alkenyl, optionally substituted alkynyl, or alkaryl;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;
about 25% to about 95% by weight of at least one filler;
about 1% to about 10% by weight of at least one disintegrant; and
about 0.1% to about 5% by weight of at least one lubricant.

2. The pharmaceutical composition according to claim 1, wherein the composition is provided as a group of individual unit dosages wherein the % RSD of the content of the antifolate compound in the group of individual unit dosages is less than about 5%.

3. The pharmaceutical composition according to claim 2, wherein the % RSD of the content of the antifolate compound in the group of individual unit dosages is less than about 4%.

4. The pharmaceutical composition according to claim 2, wherein the % RSD of the content of the antifolate compound in the group of individual unit dosages is less than about 3%.

5. The pharmaceutical composition according to claim 1, wherein the at least one filler is a microcrystalline cellulose.

6. The pharmaceutical composition according to claim 5, wherein the composition comprises at least two different types of microcrystalline cellulose.

7. The pharmaceutical composition according to claim 1, wherein the at least one filler is a starch.

8. The pharmaceutical composition according to claim 1, wherein the disintegrant comprises carboxymethyl cellulose.

9. The pharmaceutical composition according to claim 8, wherein the at least one disintegrant comprises croscarmellose sodium.

10. The pharmaceutical composition according to claim 1, wherein the at least one lubricant comprises a fatty acid or salt thereof.

11. The pharmaceutical composition according to claim 1, comprising microcrystalline cellulose, starch, croscarmellose sodium, and magnesium stearate.

12. The pharmaceutical composition according to claim 1, wherein the antifolate compound comprises a compound according to formula (7): 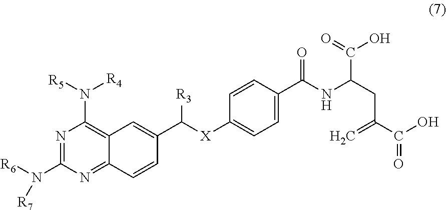
wherein:
X is CHR₈ or NR₈;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkenyl; or a pharmaceutically acceptable ester, amide, salt, solvate, enantiomer, or prodrug thereof.

13. The pharmaceutical composition according to claim 1, wherein the antifolate compound comprises a compound according to Formula (9): 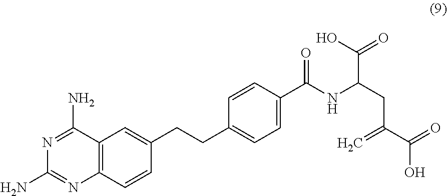
or a pharmaceutically acceptable ester, amide, salt, solvate, enantiomer, or prodrug thereof.

14. The pharmaceutical composition according to claim 1, wherein the antifolate compound comprises a compound according to Formula (11): 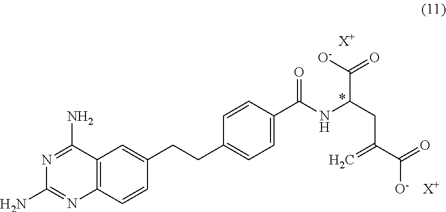
or an enantiomer thereof, wherein each X⁺ independently is a salt-forming counterion.

15. The pharmaceutical composition according to claim 14, wherein X⁺ is an alkali metal cation.

16. The pharmaceutical composition according to claim 14, wherein X⁺ is sodium.

17. The pharmaceutical composition according to claim 14, wherein X⁺ is potassium.

18. The pharmaceutical composition according to claim 14, wherein the antifolate compound is a crystalline salt.

19. The pharmaceutical composition according to claim 14, wherein the antifolate compound is a racemic salt.

20. The pharmaceutical composition according to claim 14, wherein the antifolate compound comprises a compound according to Formula (12): 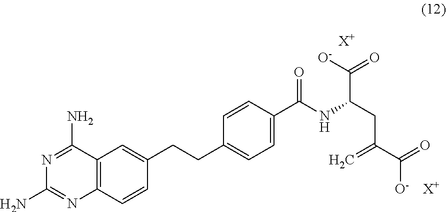
wherein each X⁺ independently is a salt-forming counterion, and wherein the antifolate compound is in the (S) enantiomeric form.

21. The pharmaceutical composition according to claim 20, wherein the antifolate compound exhibits an enantiomeric purity for the (S) enantiomer of at least about 90%.

22. The pharmaceutical composition according to claim 20, wherein the antifolate compound exhibits an enantiomeric purity for the (S) enantiomer of at least about 95%.

23. The pharmaceutical composition according to claim 20, wherein the antifolate compound exhibits an enantiomeric purity for the (S) enantiomer of at least about 99%.

24. The pharmaceutical composition according to claim 20, wherein the antifolate compound comprises a compound according to Formula (12) that is a crystalline, disodium salt in the (S) enantiomeric form exhibiting an enantiomeric purity for the (S) enantiomer of at least about 99%.

25. The pharmaceutical composition according to claim 20, wherein the antifolate compound comprises a compound according to Formula (12) that is a crystalline, dipotassium salt in the (S) enantiomeric form exhibiting an enantiomeric purity for the (S) enantiomer of at least about 99%.

26. The pharmaceutical composition according to claim 1, wherein said composition is prepared via wet granulation.

27. The pharmaceutical composition according to claim 1, wherein the antifolate compound is an alkali metal salt of (S)-2-{-4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%.

28. The pharmaceutical composition according to claim 27, wherein the salt is in a crystalline form.

29. A method for treating a condition selected from the group consisting of abnormal cell proliferation, inflammation, asthma, and arthritis, said method comprising administering to a subject in need of treatment a pharmaceutical composition according to claim 1.

30. A pharmaceutical composition comprising:
an alkali metal salt of (S)-2-{-4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%; and
at least one excipient selected from the group consisting of fillers, disintegrants, and lubricants;
wherein the composition is provided as a group of individual unit dosages and the % RSD of the content of the antifolate compound in the group of individual unit dosages is less than about 5%.

31. A method of making a pharmaceutical composition comprising an antifolate compound according to Formula (6): 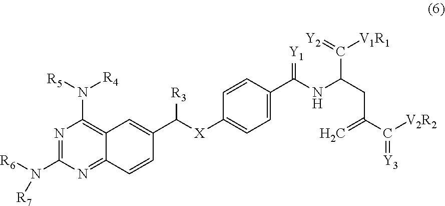
wherein:
X is CHR₈ or NR₈;
Y₁, Y₂, and Y₃ independently are O or S;
V₁ and V₂ independently are O, S, or NZ;
Z is H, optionally substituted alkyl, optionally substituted alkenyl, optionally substituted alkynyl, or alkaryl;
R₁ and R₂ independently are H, optionally substituted alkyl, optionally substituted alkenyl, optionally substituted alkynyl, or alkaryl;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;
the method comprising wet granulating about 0.01% to about 20% by weight of the antifolate compound with about 25% to about 95% by weight of at least one filler, about 1% to about 10% by weight of at least one disintegrant; and about 0.1% to about 5% by weight of at least one lubricant.

32. The method according to claim 31, wherein the antifolate compound introduced into the wet granulating method is in particulate form and has a range of particle sizes of about 10 μm to about 250 μm.

33. The method according to claim 31, said wet granulation method comprising the following steps:
a) forming a granule comprising the antifolate compound, the at least one filler, a portion of the at least one disintegrant, and a solvent;
b) at least partially drying the granule;
c) passing the granule through a granulator to form sized particles; and
d) adding the remaining portion of the at least one disintegrant and the at least one lubricant with blending.

34. The method according to claim 32, said wet granulation method comprising the following steps:
a) combining the at least one filler and a portion of the at least one disintegrant in a granulator;
b) adding the antifolate compound dissolved in a suitable solvent;
c) mixing to form a granule, optionally adding a further amount of solvent;
d) at least partially drying the granule;
e) passing the dried granule through a granulator fitted with a screen of desired size to form sized particles; and
f) adding the remaining portion of the at least one disintegrant and the at least one lubricant with blending.

35. The method according to claim 31, wherein two different fillers are used.

36. The method according to claim 35, wherein the two different fillers are two different types of microcrystalline cellulose.

37. The method according to claim 36, wherein the two different types of microcrystalline cellulose differ in particle size.

38. The method according to claim 37, wherein the first type of microcrystalline cellulose has a particle size of about 20 μm to about 125 μm and the second type of microcrystalline cellulose has a particle size of about 150 μm to about 250 μm.

39. The method according to claim 31, wherein the at least one filler is a starch.

40. The method according to claim 31, wherein the disintegrant comprises carboxymethyl cellulose.

41. The method according to claim 31, wherein the at least one lubricant comprises a fatty acid or salt thereof.

42. The method according to claim 31, wherein the antifolate compound comprises a compound according to Formula (12): 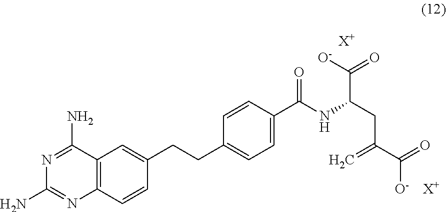
wherein each X⁺ independently is a salt-forming counterion, and wherein the antifolate compound is in the (S) enantiomeric form.

43. The method according to claim 42, wherein the antifolate compound exhibits an enantiomeric purity for the (S) enantiomer of at least about 90%.

44. A method of making a pharmaceutical composition comprising an antifolate compound according to Formula (6): 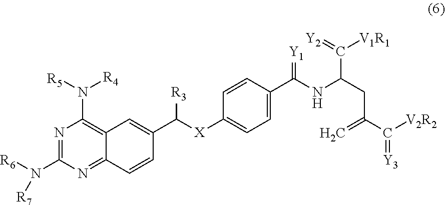
wherein:
X is CHR₈ or NR₈;
Y₁, Y₂, and Y₃ independently are O or S;
V₁ and V₂ independently are O, S, or NZ;
Z is H, optionally substituted alkyl, optionally substituted alkenyl, optionally substituted alkynyl, or alkaryl;
R₁ and R₂ independently are H, optionally substituted alkyl, optionally substituted alkenyl, optionally substituted alkynyl, or alkaryl;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;
the method comprising hot melt granulating about 0.01% to about 20% by weight of the antifolate compound with about 25% to about 95% by weight of at least one filler, about 5% to about 50% by weight of a polyglycolized glyceride; and about 0.1% to about 5% by weight of a lubricant.

45. The method according to claim 44, said hot melt granulation method comprising the following steps:
a) combining the antifolate compound with a filler;
b) adding the polyglycolized glyceride under temperature conditions sufficient to fully liquefy the polyglycolized glyceride;
c) mixing to form a granule;
d) cooling the formed granule; and
e) screening the granule and a lubricant into a container and blending the materials to form the composition.

46. The method according to claim 44, wherein the polyglycolized glyceride has a melting point that is less than about 50° C.

47. The method according to claim 44, wherein the polyglycolized glyceride has an HLB value that is greater than about 8.

48. The method according to claim 44, wherein the polyglycolized glyceride comprises a C₁₄-C₂₀ fatty acid ester.

49. The method according to claim 44, wherein the polyglycolized glyceride comprises a polyethylene glycol having a number average MW of about 1,200 to about 2,500 Da.

50. The method according to claim 44, wherein the polyglycolized glyceride and the antifolate compound are present in a weight-to-weight ratio of about 1:1 to about 50:1.

51. The method according to claim 44, wherein the filler and the polyglycolized glyceride are present in a weight-to-weight ratio of about 90:10 to about 50:50.

52. The method according to claim 44, said hot melt granulation method comprising the following steps:
a) blending the antifolate compound with a first amount of the filler to form a pre-blend;
b) combining the pre-blend with a second amount of the same or a different filler;
c) adding the polyglycolized glyceride to the combined materials under temperature conditions sufficient to fully liquefy the polyglycolized glyceride;
d) mixing the combination to form a granule;
e) cooling the formed granule;
g) screening the granule and a lubricant into a container; and
h) blending the screened materials.

53. The method according to claim 44, wherein the antifolate compound comprises a compound according to Formula (12): 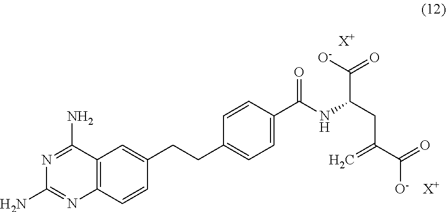
wherein each X⁺ independently is a salt-forming counterion, and wherein the antifolate compound is in the (S) enantiomeric form.

54. The method according to claim 53, wherein the antifolate compound exhibits an enantiomeric purity for the (S) enantiomer of at least about 90%.

55. The method according to claim 44, wherein the antifolate compound introduced into the hot melt granulation method is in particulate form and the particles are sized so that at least about 95% by weight of the particles pass through a 70 mesh screen.

56. A pharmaceutical composition comprising: about 0.01% to about 20% by weight of an antifolate compound according to Formula (6): 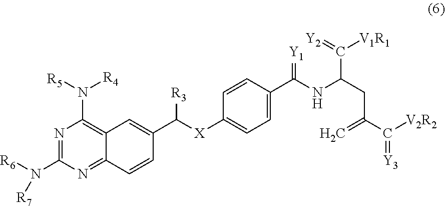
wherein:
X is CHR₈ or NR₈;
Y₁, Y₂, and Y₃ independently are O or S;
V₁ and V₂ independently are O, S, or NZ;
Z is H, optionally substituted alkyl, optionally substituted alkenyl, optionally substituted alkynyl, or alkaryl;
R₁ and R₂ independently are H, optionally substituted alkyl, optionally substituted alkenyl, optionally substituted alkynyl, or alkaryl;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;
about 25% to about 95% by weight of at least one filler;
about 5% to about 50% by weight of a polyglycolized glyceride; and
about 0.1% to about 5% by weight of a lubricant.

57. The pharmaceutical composition according to claim 56, wherein the composition is provided as a group of individual unit dosages wherein the % RSD of the content of the antifolate compound in the group of individual unit dosages is less than about 5%.

58. The pharmaceutical composition according to claim 56, wherein the polyglycolized glyceride has a melting point that is less than about 50° C.

59. The pharmaceutical composition according to claim 56, wherein the polyglycolized glyceride has an HLB value that is greater than about 8.

60. The pharmaceutical composition according to claim 56, wherein the polyglycolized glyceride comprises a C₁₄-C₂₀ fatty acid ester.

61. The pharmaceutical composition according to claim 56, wherein the polyglycolized glyceride comprises a polyethylene glycol having a number average MW of about 1,200 to about 2,500 Da.

62. The pharmaceutical composition according to claim 56, wherein the polyglycolized glyceride and the antifolate compound are present in a weight-to-weight ratio of about 1:1 to about 50:1.

63. The pharmaceutical composition according to claim 56, wherein the filler and the polyglycolized glyceride are present in a weight-to-weight ratio of about 90:10 to about 50:50.

64. The pharmaceutical composition according to claim 56, wherein the antifolate compound comprises a compound according to formula (7): 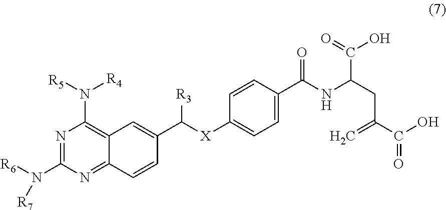
wherein:
X is CHR₈ or NR₈;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkenyl; or a pharmaceutically acceptable ester, amide, salt, solvate, enantiomer, or prodrug thereof.

65. The pharmaceutical composition according to claim 56, wherein the antifolate compound comprises a compound according to Formula (9): 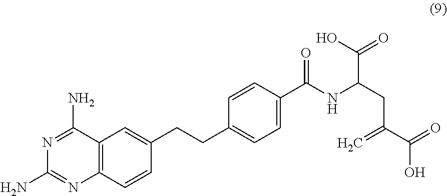
or a pharmaceutically acceptable ester, amide, salt, solvate, enantiomer, or prodrug thereof.

66. The pharmaceutical composition according to claim 56, wherein the antifolate compound comprises a compound according to Formula (11): 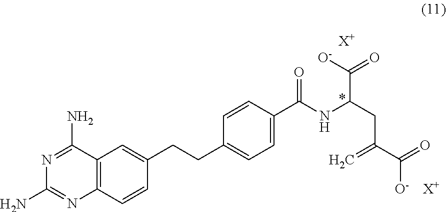
or an enantiomer thereof, wherein each X⁺ independently is a salt-forming counterion.

67. The pharmaceutical composition according to claim 66, wherein X⁺ is an alkali metal cation.

68. The pharmaceutical composition according to claim 66, wherein X⁺ is sodium.

69. The pharmaceutical composition according to claim 66, wherein X⁺ is potassium.

70. The pharmaceutical composition according to claim 66, wherein the antifolate compound is a crystalline salt.

71. The pharmaceutical composition according to claim 66, wherein the antifolate compound is a racemic salt.

72. The pharmaceutical composition according to claim 66, wherein the antifolate compound comprises a compound according to Formula (12): 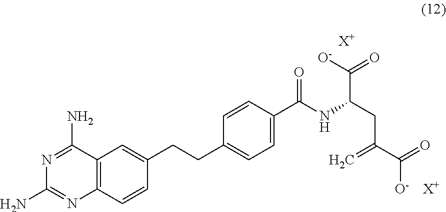
wherein each X⁺ independently is a salt-forming counterion, and wherein the antifolate compound is in the (S) enantiomeric form.

73. The pharmaceutical composition according to claim 72, wherein the antifolate compound exhibits an enantiomeric purity for the (S) enantiomer of at least about 90%.

74. The pharmaceutical composition according to claim 72, wherein the antifolate compound exhibits an enantiomeric purity for the (S) enantiomer of at least about 95%.

75. The pharmaceutical composition according to claim 72, wherein the antifolate compound exhibits an enantiomeric purity for the (S) enantiomer of at least about 99%.

76. The pharmaceutical composition according to claim 72, wherein the antifolate compound comprises a compound according to Formula (12) that is a crystalline, disodium salt in the (S) enantiomeric form exhibiting an enantiomeric purity for the (S) enantiomer of at least about 99%.

77. The pharmaceutical composition according to claim 72, wherein the antifolate compound comprises a compound according to Formula (12) that is a crystalline, dipotassium salt in the (S) enantiomeric form exhibiting an enantiomeric purity for the (S) enantiomer of at least about 99%.

78. The pharmaceutical composition according to claim 56, wherein the antifolate compound is an alkali metal salt of (S)-2-{4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%.

79. A method for treating a condition selected from the group consisting of abnormal cell proliferation, inflammation, asthma, and arthritis, said method comprising administering to a subject in need of treatment a pharmaceutical composition according to claim 56.
